(19) United States
(12) Patent Application Publication
(10) Pub. No.: US 2005/0261327 A1
(43) Pub. Date: Nov. 24, 2005

(54) 2-(Bicyclo)alkylamino-derivatives as mediatores of chronic pain and inflammation

(76) Inventors: Mark G. Bock, Hatfield, PA (US); Scott D. Kuduk, Harleysville, PA (US); Dai-Shi Su, Dresher, PA (US); Michael R. Wood, Harleysville, PA (US)

Correspondence Address: MERCK AND CO., INC, P O BOX 2000, RAHWAY, NJ, 07065-0907, US

(21) Appl. No.: 11/131,642

(22) Filed: May 18, 2005

Related U.S. Application Data
(60) Provisional application No. 60/572,708, filed on May 20, 2004.

(51) Int. Cl. A61K 31/4747; C07D 487/10

(52) U.S. Cl. 514/278; 514/409; 546/16

(57) ABSTRACT

Compounds disclosed herein are bradykinin B1 antagonist compounds useful in the treatment or prevention of symptoms such as pain and inflammation associated with the bradykinin B1 pathway.

### BACKGROUND OF THE INVENTION

This invention is directed to 2-(bicyclo)alkylamino derivatives as mediators of chronic pain and iflammation. In particular, this invention is directed to 2-(bicyclo)alkylamino derivatives that are bradykinin antagonists or inverse agonists.

Bradykinin (“BK”) is a kinin which plays an important role in the pathophysiological processes accompanying acute and chronic pain and inflammation. Bradykinin (BK), like other kinins, is an autacoid peptide produced by the catalytic action of kallikrein enzymes on plasma and tissue precursors termed kininogens. The biological actions of BK are mediated by at least two major G-protein-coupled BK receptors termed B1 and B2. It is generally believed that B2 receptors, but not B1 receptors, are expressed in normal tissues and that inflammation, tissue damage or bacterial infection can rapidly induce B1 receptor expression. This makes the B1 receptor a particularly attractive drug target. The putative role of kinins, and specifically BK, in the management of pain and inflammation has provided the impetus for developing potent and selective BK antagonists. In recent years, this effort has been heightened with the expectation that useful therapeutic agents with analgesic and anti-inflammatory properties would provide relief from maladies mediated through a BK receptor pathway (see e.g., M. G. Bock and J. Longmore, Current Opinion in Chem. Biol., 4:401-406 (2000)). Accordingly, there is a need for novel compounds that are effective in blocking or reversing activation of bradykinin receptors. Such compounds would be useful in the management of pain and inflammation, as well as in the treatment or prevention of diseases and disorders mediated by bradykinin; further, such compounds are also useful as research tools (in vivo and in vitro).

### SUMMARY OF THE INVENTION

The present invention provides Compounds of Formula I, Formula II and Formula III which are bradykinin antagonists or inverse agonists, pharmaceutical compositions containing such compounds, and methods of using them as therapeutic agents.

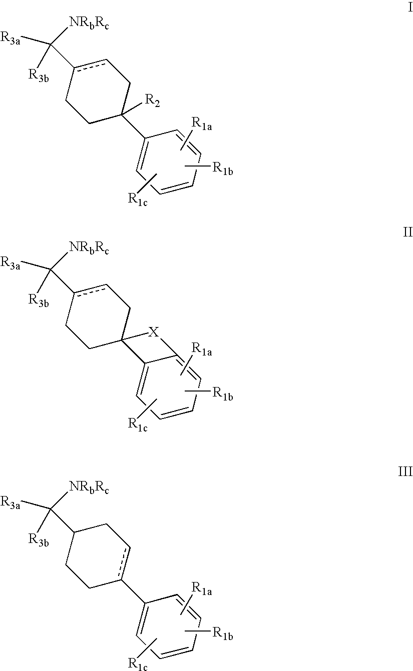

### DETAILED DESCRIPTION OF THE INVENTION

The present invention provides compounds of Formula I, Formula II and Formula III: 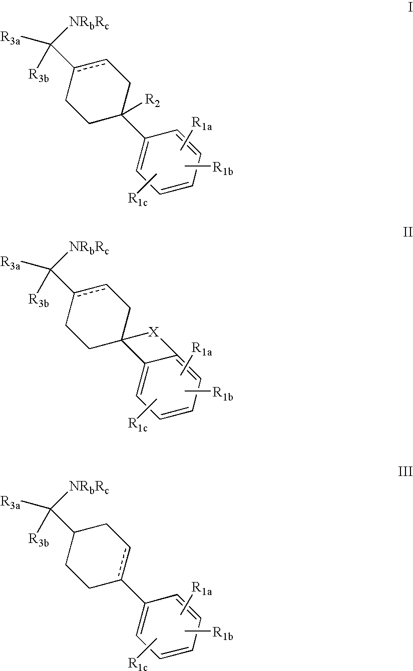
or a pharmaceutically acceptable salt thereof wherein
R^{1a}, R_{1b} and R_{1c} are each selected from
   (1) hydrogen,
   (2) —C₁₋₈ alkyl, optionally substituted with 1, 2, 3, 4 or 5 groups independently selected from halogen, nitro, cyano, —COR^{a}, —CO₂R^{a}, —CONR^{d}R^{e}, —OR^{a}, —OC(O)R^{a}, —SOₘR^{a′}, —NR^{d}R^{e}, —NR^{d}C(O)R^{a}, —NR^{d}SO₂R^{a′}, —NR^{d}CO₂R^{a},
   (3) —C₃₋₈ cycloalkyl,
   (4) —C₂₋₈ alkenyl optionally substituted with CO₂R^{a},
   (5) halogen,
   (6) cyano,
   (7) nitro,
   (8) —NR^{d}R^{e},
   (9) —NR^{d}C(O)R^{a},
   (10) —NR^{d}CO₂R^{a},
   (11) —NR^{d}C(O)NR^{d}R^{e},
   (12) —NR^{d}C(O)NR^{d}CO₂R^{a},
   (13) —NR^{d}SO₂R^{a′},
   (14) —CO₂R^{a},
   (15) —COR^{a},
   (16) —C(O)NR^{d}R^{e},
   (17) —C(O)NHOR^{a},
   (18) —C(═NOR^{a})R^{a},
   (19) —C(═NOR^{a})NR^{d}R^{e},
   (20) —OR^{a},
   (21) —OC(O)R^{a},
   (22) —S(O)ₘR^{a}′, wherein R^{a}′, is a non-hydrogen group selected from R^{a},
   (23) —SO₂NR^{d}R^{e},
   (24) substituted or unsubstituted heterocycle (such as oxadiazole, tetrazole, triazole, pyrazole, oxazole, isoxazole, thiazole, 4,5-dihydro-oxazole, 4,5-dihydro-1,2,4-oxadiazol-5-one), wherein the heterocycle is (a) a 5-membered aromatic ring having a ring heteroatom selected from N, O and S, and optionally having up to 3 additional ring nitrogen atoms wherein said ring is optionally benzo-fused; (b) a 6-membered aromatic ring containing from 1 to 3 ring nitrogen atoms and N-oxides thereof, wherein said ring is optionally benzo-fused; and (c) a 5- or 6-membered non-aromatic heterocyclic ring and wherein the substituents are 1, 2 or 3 groups independently selected from C₁₋₄alkyl optionally substituted with 1, 2 or 3 halogen atoms, —OR^{a}, or —OC(O)R^{a},
with the proviso that not more than one of R₁ₐ, R_{1b} and R_{1c} is a heterocycle;
R² is selected from
   (1) H,
   (2) —C₁₋₆ alkyl optionally substituted with 1, 2 or 3 halogen atoms,
   (3) —C₃₋₇ cycloalkyl optionally containing 1 or 2 ring members selected from O and N—R^{d} group,
   (4) —(CH₂)ₙ—C(O)—C₁₋₆ alkyl optionally substituted with 1, 2 or 3 halogen atoms,
   (5) —(CH₂)ₙOR^{a},
   (6) —(CH₂)ₙS(O)ₘR^{a′},
   (7) —(CH₂)ₙNR^{d}R^{e},
   (8) —(CH₂)ₙC(O)OR^{a},
   (9) —(CH₂)ₙOCOR^{a′},
   (10) —(CH₂)ₙNR^{d}C(O)R^{a′},
   (11) —(CH₂)ₙNS(O)ₘR^{a′},
   (12) —(CH₂)ₙC(O)NR^{d}R^{e},
   (13) —(CH₂)ₙCN,
   (14) —(CH₂)ₙ-AR, wherein AR is selected from benzene, naphthalene, indole, indoline, is optionally substituted with 1, 2, 3 or 4 groups independently selected from halogen, C₁₋₄ alkyl optionally substituted with 1, 2, 3, 4 or 5 halogen atoms,
   (15) —(CH₂)ₙ—NO2,
   (16) —(CH₂)ₙ-heterocycle, wherein the heterocycle is an optionally substituted (a) 5-membered ring having a ring heteroatom selected from N, O and S, and optionally having up to 3 additional ring nitrogen atoms wherein said ring is optionally benzo-fused; (b) 6-membered ring containing from 1 to 3 ring nitrogen atoms and N-oxides thereof, wherein said ring is optionally benzo-fused; and (c) 5- or 6-membered non-aromatic heterocyclic ring, and wherein the substituents on the heterocycle are with 1, 2 or 3 groups independently selected from —C₁₋₄ alkyl optionally substituted with 1 to 5 halogen atoms, —OR^{a} or —OC(O)R^{a};
R^{3a} and R^{3b} are selected from
   (1) Hand
   (2) —C₁₋₄ alkyl optionally substituted with 1, 2 or 3 halogen atoms;
R^{a} is independently selected from:
   (1) H,
   (2) —C₁₋₆ alkyl optionally substituted with 1, 2 or 3 halogen atoms,
   (3) —C₃₋₇ cycloalkyl ring, the ring optionally containing 1 or 2 ring members selected from O and N—R^{d} group,
   (4) —C(O)—C₁₋₆ alkyl optionally substituted with 1, 2 or 3 halogen atoms,
   (5) AR1, wherein AR1 is selected from benzene, pyridine, thiophene, naphthalene, indole, indoline, pyrimidine, imidazole, optionally substituted with 1, 2, 3 or 4 groups independently selected from halogen, C₁₋₄ alkyl optionally substituted with 1, 2, 3, 4 or 5 halogen atoms, hydroxy, C₁₋₄ alkoxy optionally substituted with 1, 2, 3, 4 or 5 halogen atoms,
   (6) nitro,
   (7) cyano, and
   (8) —NR^{d}R^{e};
R^{a′} is independently selected from:
   (1) —C₁₋₆ alkyl optionally substituted with 1, 2 or 3 halogen atoms,
   (2) —C₃₋₇ cycloalkyl optionally containing ring 0 and/or N—R^{d} group,
   (3) —C(O)—C₁₋₆ alkyl optionally substituted with 1, 2 or 3 halogen atoms,
   (4) AR1, wherein AR1 is selected from benzene, pyridine, thiophene, naphthalene, indole, indoline, pyrimidine, imidazole optionally substituted with 1, 2, 3 or 4 groups independently selected from halogen, C₁₋₄ alkyl optionally substituted with 1, 2, 3, 4, or 5 halogen atoms,
   (5) hydroxy,
   (6) —C₁₋₄ alkoxy optionally substituted with 1, 2, 3, 4 or 5 halogen atoms,
   (7) nitro,
   (8) cyano, and
   (9) —NR^{d}R^{e};
R^{b} and R^{f} are each independently selected from:
   (1) H,
   (2) —C₁₋₆ alkyl optionally substituted with 1, 2 or 3 halogen atoms,
R^{c} is independently selected from:
   (1) hydrogen,
   (2) —C₁₋₆ alkyl optionally substituted with 1, 2 or 3 halogen atoms,
   (3) —C₃₋₇ cycloalkyl optionally containing 1 or 2 ring members selected from O and N—R^{e} group,
   (4) 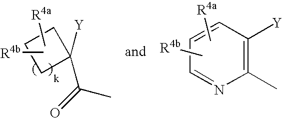
R^{4a} and R^{4b} are independently selected form
   (1) H,
   (2) halogen, and
   (3) —C₁₋₄ alkyl optionally substituted with 1, 2, 3 or 4 groups selected from halogen, —OR^{a}, —OC(O)R^{a}, —S(O)ₘR^{a′}, —OS(O)₂R^{a′}, and —NR^{d}R^{e};
X is selected from
   (1) —(CH₂)ₙN[S(O)₂R^{a′}](CH₂)ₙ—,
   (2) —(CH₂)ₙN[C(O)R^{a}](CH₂)ₙ—,
   (3) —(CH₂)ₙN [C(O)OR^{a}](CH₂)ₙ—,
   (4) —(CH₂)ₙNR^{d}(CH₂)ₙ—,
   (5) —(CH₂)ₙOC(O)(CH₂)ₙ—,
   (6) —(CH₂)ₙC(O)NR^{d}(CH₂)ₙ—,
   (7) —(CH₂)ₙC(O)O(CH₂)ₙ—,
   (8) —(CH₂)ₙNR^{d}C(O)(CH₂)ₙ—,
   (9) —(CH₂)ₙNS(O)₂(CH₂)ₙ—,
   (10) —(CH₂)ₙS(O)₂N(CH₂)ₙ—,
   (11) —(CH₂)ₙS(O)₂O(CH₂)ₙ—,
   (12) —(CH₂)ₙOS(O)₂(CH₂)ₙ—,
   (13) —(CH₂)ₙOS(O)₂O(CH₂)ₙ—,
   (14) —(CH₂)ₙNS(O)₂O(CH₂)ₙ—,
   (15) —(CH₂)ₙOS(O)₂N(CH₂)ₙ—,
   (16) —(CH₂)ⱼ—,
   (17) —(CH₂)ₙO(CH₂)ₙ—,
   (18) —(CH₂)ₙO(CH₂)ⱼO(CH₂)ₙ—, and
   (19) —(CH₂)ₙCH═CH(CH₂)ₙ—;
Y is selected from
   (1) —NR^{b}C(O)R⁵,
   (2) —NR^{d}C(O)NR^{d}R^{e},
   (3) —OC(O)NR^{d}R^{e},
   (4) —NR^{d}S(O)₂NR^{d}R^{e},
   (5) —NR^{d}C(O)OR^{a},
R^{d} and R^{e} are each independently selected from:
   (1) H,
   (2) —C₁₋₆ alkyl optionally substituted with 1 to 3 halogen atoms,
   (3) —C₃₋₇ cycloalkyl optionally containing ring 0 and/or N—R^{b} group
   (4) —(CH₂)ₙNR^{b}R^{f},
   (5) —(CH₂)ₙOR^{a},
   (6) —(CH₂)ₙS(O)ₘR^{a′},
   (7) —(CH₂)ₙC(O)OR^{a},
   (8) —(CH₂)ₙC(O)NR^{b}R^{f}
   (9) —C(O)R^{f},
   (10) —S(O)₂R_{f}
   (11) AR2,
   (12) -AR2-C₁₋₄alkyl,
   (13) —C₁₋₄-alkyl-AR2,
wherein AR2, -AR2-C₁₋₄alkyl, and —C₁₋₄-alkyl-AR2 are optionally substituted with 1, 2, 3 or 4 groups independently selected from halogen, —C₁₋₄ alkyl optionally substituted with 1, 2, 3, 4 or 5 halogen atoms, hydroxy, —C₁₋₄ alkoxy optionally substituted with 1, 2, 3, 4 or 5 halogen atoms, nitro, cyano and —NR^{b}R^{c}, and wherein AR2 is selected from benzene, pyridine, thiophene, naphthalene, indene, indan, thiodiazole, benzofuran, indole, indoline, benzothiophene, pyrimidine, triazine, thioazole, isoxazole, oxazole, benzimidazole, imidazole;
R⁵ is selected from
   (1) —C₁₋₆ alkyl optionally substituted with 1, 2, 3, 4 or 5 groups independently selected from halogen, nitro, cyano, —OR^{a}, —SR^{a}, —COR^{a}, —SO₂R^{a′}, —CO₂R^{a}, —OC(O)R^{a}, —NR^{d}R^{e}, —NR^{d}C(O)R^{a}, —NR^{d}C(O)₂R^{a}, —C(O)NR^{d}R^{e}, —C₃₋₈ cycloalkyl,
   (2) —C₃₋₈ cycloalkyl optionally substituted with 1 to 5 groups independently selected from halogen, nitro, cyano and phenyl,
   (3) —C₃₋₆ alkynyl,
   (4) —C₂₋₆ alkenyl optionally substituted with hydroxyethyl,
   (5) —(CH₂)ₙ-AR4 optionally substituted with 1 to 3 groups independently selected from halogen, nitro, cyano, —OR^{a}, —SR^{a}, —C(O)₂R^{a}, —C₁₋₄ alkyl and —C₁₋₃ haloalkyl, wherein AR4 is selected from phenyl, 3,4-methylenedioxyphenyl and naphthyl;
   (6) —(CH₂)ₙ-heterocycle optionally substituted with 1, 2 or 3 groups independently selected from halogen, nitro, cyano, OR^{a}, SR^{a}, C₁₋₄ alkyl and C₁₋₃ haloalkyl wherein said heterocycle is selected from (a) a 5-membered ring having a ring heteroatom selected from N, O and S, and optionally having up to 3 additional ring nitrogen atoms wherein said ring is optionally benzo-fused; (b) a 6-membered ring containing from 1 to 3 ring nitrogen atoms and N-oxides thereof, wherein said ring is optionally benzo-fused; and (c) a 5- or 6-membered non-aromatic heterocyclic ring selected from tetrahydrofuranyl, 5-oxotetrahydrofuranyl, 2-oxo-2H-pyranyl, 6-oxo-1,6-dihydropyridazinyl,
   (7) —C(O)₂R^{a}, and
   (8) —C(O)NR^{d}R^{e};
n is 0, 1, 2, 3 or 4;
m is 0, 1 or 2;
j is 1, 2, 3 or 4,
k is 0 or 1.

In one aspect are the compounds wherein
R₁ₐ is selected from
   (1) hydrogen,
   (2) —C₁₋₈ alkyl, optionally substituted with 1, 2, 3, 4 or 5 groups independently selected from halogen, nitro, cyano, COR^{a}, CO₂R^{a}, CONR^{d}R^{e}, OR^{a}, OC(O)R^{a}, SOₘR^{a′}, NR^{d}R^{e}, NR^{d}C(O)R^{a}, NR^{d}SO₂R^{a′}, NR^{d}CO₂R^{a},
   (1) halogen,
   (2) cyano,
   (3) —CO₂R^{a},
   (4) —COR^{a},
   (5) —OR^{a},
   (6) —OC(O)R^{a},
   (7) substituted or unsubstituted heterocycle where the heterocycle is selected from oxadiazole, tetrazole, triazole, pyrazole, oxazole, isoxazole, thiazole, 4,5-dihydro-oxazole, 4,5-dihydro-1,2,4-oxadiazol-5-one, and wherein said substituent is 1, 2 or 3 groups independently selected from C₁₋₄alkyl optionally substituted with 1, 2 or 3 halogen atoms, —OR^{a}, or —OC(O)R^{a}, with the proviso that not more than one of R^{1a}, R^{1b} and R^{1c} is a heterocycle.

In one aspect are the compounds wherein
R² is selected from
   (1) H,
   (2) —C₁₋₆ alkyl optionally substituted with 1, 2 or 3 halogen atoms,
   (3) —C(O)—C₁₋₆ alkyl optionally substituted with 1, 2 or 3 halogen atoms,
   (4) —(CH₂)ₙOR^{a},
   (5) —(CH₂)ₙNR^{d}R^{e},
   (6) —(CH₂)ₙC(O)NR^{d}R^{e},
   (7) —(CH₂)ₙ CN,
   (8) —(CH₂)ₙ-AR, wherein AR is optionally substituted with 1, 2, 3 or 4 groups independently selected from halogen, C₁₋₄ alkyl optionally substituted with 1, 2, 3, 4 or 5 halogen atoms,
   (9) C₁₋₄ alkoxy optionally substituted with 1, 2, 3, 4 or 5 halogen atoms,
   (10) —(CH₂)ₙ-heterocycle, where the heterocycle is a 5-membered ring having a ring heteroatom selected from N, O and S, and optionally having 1, 2 or 3 additional ring nitrogen atoms, 4,5-dihydro-oxazolyl and 4,5-dihydro-1,2,4-oxadiazolyl, and wherein said substituent is 1, 2 or 3 groups independently selected from —C₁₋₄ alkyl optionally substituted with 1 to 5 halogen atoms, —OR^{a} or —OC(O)R^{a}.

In one aspect are the compounds wherein
R^{a} and R^{a′} are each independently selected from:
   (1) H,
   (2) —C₁₋₆ alkyl optionally substituted with 1, 2 or 3 halogen atoms,
   (3) AR1, wherein AR1 is optionally substituted with 1, 2, 3 or 4 groups independently selected from halogen, C₁₋₄ alkyl optionally substituted with 1, 2, 3, 4 or 5 halogen atoms, hydroxy, C₁₋₄ alkoxy optionally substituted with 1, 2, 3, 4 or 5 halogen atoms,
   (4) cyano, and
   (5) —NR^{d}R^{e}.

In one aspect are the compounds wherein
R^{c} is independently selected from:
   (1) hydrogen,
   (2) —C₁₋₆ alkyl optionally substituted with 1, 2 or 3 halogen atoms,
   (3)

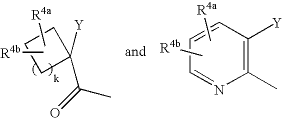

In one aspect are the compounds wherein
X is selected from:
   (1) —CH₂NH,
   (2) —OC(O)—,
   (3) —C(O)NH—,
   (4) —OCH₂CH₂—,
   (5) —OC(O)—,
   (6) —OCH₂—,
   (7) —OCH₂OCH₂—,
   (8) —OCHCH—, and
   (9) —NHC(O)—.

In one aspect are the compounds wherein
Y is —NR^{b}C(O)R⁵.

In one aspect are the compounds wherein
R^{d} is independently selected from:
   (1) H,
   (2) —C₁₋₆ alkyl optionally substituted with 1 to 3 halogen atoms,
   (3) —(CH₂)ₙOR^{a},
   (4) —C(O)R^{e},
   (5) —S(O)₂R^{e}.

In one aspect are the compounds wherein
R⁵ is —C₁₋₆ alkyl optionally substituted with 1, 2, 3, 4 or 5 groups independently selected from halogen, nitro, cyano, —OR^{a}, —SR^{a}, —COR^{a}, —SO₂R^{a′}, —CO₂R^{a}, —OC(O)R^{a}, —NR^{d}R^{e}, —NR^{d}C(O)R^{a}, —NR^{d}C(O)₂R^{a}, —C(O)NR^{d}R^{e}, —C₃₋₈ cycloalkyl.

In one aspect the invention is directed to Compounds of Formula I 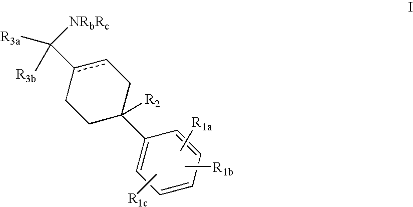
and pharmaceutically acceptable salts thereof wherein R_{b} is H and R_{c} is:

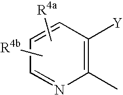

Within this aspect there is the genus of compounds of Formula Ia 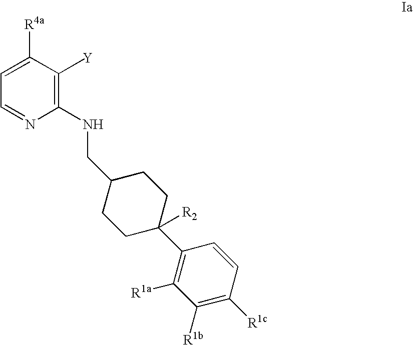
and pharmaceutically acceptable salts.

Within this genus there is the subgenus of compounds wherein:
R² is H, CN, OH, —CH₃, —CH₂OH, —C(O)NH₂, —CO₂CH₃, —OC(O)CH₃, phenyl and oxadiazole.

Within this genus there is another subgenus of compounds wherein:
R^{a4} is selected from hydrogen, halo and CH₃.

Within this genus there is another subgenus of compounds wherein:
R^{1a}, R^{1b} and R^{1c} are each independently selected from the group consisting of hydrogen, halo, —CF₃, —OCH₃, —OCF₃, —CO₂CH₃ and morpholine.

Within this genus there is another subgenus of compounds wherein:
Y is —NHC(O)R⁵, and
R⁵ is selected from the group consisting of CH₂CF₃, CH₂CN and isoxazole.

Within this genus there is another subgenus of compounds wherein:
R² is H, CN, OH, CH₃, CH₂OH, C(O)NH₂, CO₂CH₃, OC(O)CH₃, phenyl and oxadiazole,
R^{a4} is selected from hydrogen and CH₃,
R^{1a}, R^{1b} and R^{1c} are each independently selected from the group consisting of hydrogen, halo, CF₃, OCH₃, OCF₃, CO₂CH₃ and morpholine,
Y is NHC(O)R⁵, and
R⁵ is selected from the group consisting of —CH₂CF₃, —CH₂CN and isoxazole.
   Within this subgenus there is a class of compounds wherein:
R² is H or CN.

In a second aspect the invention is directed to Compounds of Formula I 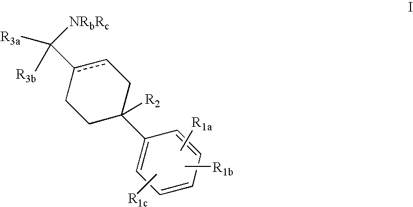
and pharmaceutically acceptable salts thereof wherein R_{c} is:

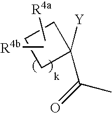

Within this aspect there is a genus of compounds of Formula Ib

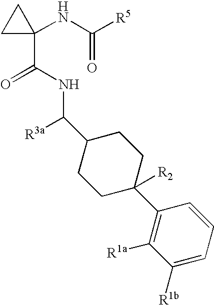

Within this genus there is the subgenus of compounds wherein:
R² is H, CN, OH, —CH₃, —CH₂OH, —C(O)NH₂, —CO₂CH₃, —OC(O)CH₃, phenyl and oxadiazole.

Within this subgenus there is a class of compounds wherein R² is CN.

Within this genus there is another subgenus of compounds wherein:
R^{1a} and R^{1b} are each independently selected from the group consisting of hydrogen, halo, —CF₃, —OCH₃, —OCF₃ and —CO₂CH₃.

Within this genus there is another subgenus of compounds wherein:
R^{3a} is selected from the group consisting of hydrogen and methyl.

Within this genus there is another subgenus of compounds wherein:
R⁵ is selected from the group consisting of —CH₂CF₃, —CH₂CN, —C(O)CH₃, —C(O)NHCH₃, C(O)NHC(O)OCH₃, pyridyl optionally substituted with NO₂, isoxazole, and pyrimidine.

Within this genus there is the subgenus of compounds wherein:
R² is CN,
R^{1a} and R^{1b} are each independently selected from the group consisting of hydrogen, halo, —CF₃, —OCH₃, —OCF₃ and —CO₂CH₃,
R^{3a} is selected from the group consisting of hydrogen and methyl, and
R⁵ is selected from the group consisting of —CH₂CF₃, —CH₂CN, —C(O)CH₃, —C(O)NHCH₃, —C(O)NHC(O)OCH₃, pyridyl optionally substituted with NO₂, isoxazole, and pyrimidine.

In another aspect the invention is directed to compounds of Formula Ib

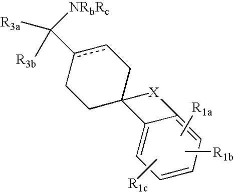

Within this aspect is the genus of compounds and pharmaceutically acceptable salts of the Formula Ib′ and Ib″

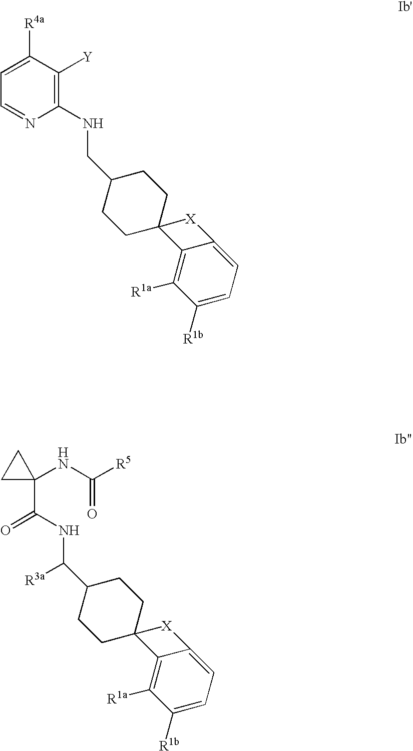

Within this genus is a subgenus of compounds wherein
R^{1a} and R^{1b} are each independently selected from the group consisting of hydrogen and halo.

Within this genus is the subgenus of compounds wherein
R³ is hydrogen.

Within this genus is the subgenus of compounds wherein
R^{4a} is hydrogen or —CH₃.

Within this genus there is another subgenus of compounds wherein:
Y is NHC(O)R⁵, and
R⁵ is selected from the group consisting of —CH₂CF₃, —CH₂CN, furan and pyrimidine.

Within this genus there is another subgenus of compounds wherein
X is selected from:
   (1) —CH₂N[S(O)₂CH₃]—,
   (2) —CH₂NC(O)CF₃,
   (3) —CH₂NC(O)OCH₃,
   (4) —CH₂NC(O)OC(CH₃)₃,
   (5) —CH₂NH,
   (6) —CH₂NC(O)CH₃,
   (7) —CH₂NCH₃,
   (8) —OC(O)—,
   (9) —CH₂NCH₂CF₃,
   (10) —C(O)NH—,
   (1) —OCH₂CH₂—,
   (12) —OC(O)—,
   (13) —CH₂CH₂—,
   (14) —OCH₂—,
   (15) —OCH₂OCH₂—
   (16) —OCHCH—, and
   (17) —NHC(O)—.

Within this subgenus there is another class of compounds wherein
X is selected from:
   (1) —CH₂NH—,
   (2) —OC(O)—,
   (3) —C(O)NH—,
   (4) —OCH₂CH₂—,
   (5) —OC(O)—,
   (6) —OCH₂—,
   (7) —OCH₂OCH₂—,
   (8) —OCHCH—, and
   (9) —NHC(O)—.

Within this aspect is an genus of compounds wherein
R^{1a} and R^{1b} are each independently selected from the group consisting of hydrogen and halo;
R³ is hydrogen;
R^{4a} is hydrogen or —CH₃;
Y is NHC(O)R⁵;
R⁵ is selected from the group consisting of —CH₂CF₃, —CH₂CN, furan and pyrimidine; and
X is selected from:
   (1) —CH₂NH,
   (2) —OC(O)—,
   (3) —C(O)NH—,
   (4) —OCH₂CH₂—,
   (5) —OC(O)—,
   (6) —OCH₂—,
   (7) —OCH₂OCH₂—,
   (8) —OCHCH—, and
   (9) —NHC(O)—.
      Unless otherwise stated, the following terms have the meanings indicated below:

“Alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like.

“Alkenyl” means a linear or branched carbon chain containing at least one C═C bond. Examples of alkenyl include allyl, 2-butenyl, 3-butenyl, 1-methyl-2-propenyl, and the like.

“Aryl” means phenyl or naphthyl.

“Halogen” means fluorine, chlorine, bromine and iodine.

“Optionally substituted” is intended to include both substituted and unsubstituted. Thus, for example, optionally substituted aryl could represent a pentafluorophenyl or a phenyl ring.

Optical Isomers—Diastereomers—Geometric Isomers—Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I, II and III are shown without a definitive stereochemistry at certain positions. The present invention includes all stereoisomers of and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I, II and III may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I, II and III.

Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

Prodrugs

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

Pharmaceutical Compositions

Another aspect of the present invention provides pharmaceutical compositions which comprises a compound of Formula I, II and III and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, II and III, additional active ingredient(s), and pharmaceutically acceptable excipients.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, II and III, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, II and III, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I, II and III. The compounds of Formula I, II and III, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I, II and III of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The following are examples of representative pharmaceutical dosage forms for the compounds of Formula I:

| Injectable Suspension (I.M.) | mg/mL |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |

| Water for injection to a total volume of 1 mL | |
|---|---|
| Tablet | mg/tablet |
| Compound of Formula I | 25 |
| Microcrystalline Cellulose | 415 |
| Povidone | 14.0 |
| Pregelatinized Starch | 43.5 |
| Magnesium Stearate | 2.5 |
| | 500 |

| Capsule | mg/capsule |
|---|---|
| Compound of Formula I | 25 |
| Lactose Powder | 573.5 |
| Magnesium Stearate | 1.5 |
| | 600 |

Utilities

Compounds of this invention are antagonists or inverse agonists of bradykinin receptor, in particular the bradykinin B1 receptor, and as such are useful in the treatment and prevention of diseases and conditions mediated through the bradykinin receptor pathway such as pain and inflammation. The compounds would be effective in the treatment or prevention of pain including, for example, visceral pain (such as pancreatitis, interstitial cystitis, renal colic), neuropathic pain (such as postherpetic neuralgia, nerve injury, the “dynias”, e.g., vulvodynia, phantom limb pain, root avulsions, painful traumatic mononeuropathy, painful polyneuropathy), central pain syndromes (potentially caused by virtually any lesion at any level of the nervous system), and postsurgical pain syndromes (eg, postmastectomy syndrome, postthoracotomy syndrome, stump pain)), bone and joint pain (osteoarthritis), repetitive motion pain, dental pain, cancer pain, myofascial pain (muscular injury, fibromyalgia), perioperative pain (general surgery, gynecological), chronic pain, dysmennorhea, as well as pain associated with angina, and inflammatory pain of varied origins (e.g. osteoarthritis, rheumatoid arthritis, rheumatic disease, teno-synovitis and gout).

Further, the compounds of this invention can also be used to treat hyperreactive airways and to treat inflammatory events associated with airways disease e.g. asthma including allergic asthma (atopic or non-atopic) as well as exercise-induced bronchoconstriction, occupational asthma, viral- or bacterial exacerbation of asthma, other non-allergic asthmas and “wheezy-infant syndrome”. Compounds of the present invention may also be used to treat chronic obstructive pulmonary disease including emphysema, adult respiratory distress syndrome, bronchitis, pneumonia, allergic rhinitis (seasonal and perennial), and vasomotor rhinitis. They may also be effective against pneumoconiosis, including aluminosis, anthracosis, asbestosis, chalicosis, ptilosis, siderosis, silicosis, tabacosis and byssinosis.

Compounds of the present invention may also be used for the treatment of inflammatory bowel disease including Crohn's disease and ulcerative colitis, irritable bowel syndrome, pancreatitis, nephritis, cystitis (interstitial cystitis), uveitis, inflammatory skin disorders such as psoriasis and eczema, rheumatoid arthritis and edema resulting from trauma associated with burns, sprains or fracture, cerebral edema and angioedema. They may be used to treat diabetic vasculopathy, diabetic neuropathy, diabetic retinopathy, post capillary resistance or diabetic symptoms associated with insulitis (e.g. hyperglycemia, diuresis, proteinuria and increased nitrite and kallikrein urinary excretion). They may be used as smooth muscle relaxants for the treatment of spasm of the gastrointestinal tract or uterus. Additionally, they may be effective against liver disease, multiple sclerosis, cardiovascular disease, e.g. atherosclerosis, congestive heart failure, myocardial infarct; neurodegenerative diseases, eg. Parkinson's and Alzheimers disease, epilepsy, septic shock e.g. as anti-hypovolemic and/or anti-hypotensive agents, headache including cluster headache, migraine including prophylactic and acute use, closed head trauma, cancer, sepsis, gingivitis, osteoporosis, benign prostatic hyperplasia and hyperactive bladder. Animal models of these diseases and conditions are generally well known in the art, and may be suitable for evaluating compounds of the present invention for their potential utilities. Finally, compounds of the present invention are also useful as research tools (in vivo and in vitro).

The compounds of this invention are useful in the treatment of pain and inflammation by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

The compounds would be effective in the treatment or prevention of pain including, for example, bone and joint pain (osteoarthritis), repetitive motion pain, dental pain, cancer pain, myofascial pain (muscular injury, fibromyalgia), perioperative pain (general surgery, gynecological) and chronic pain by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

In particular, inflammatory pain such as, for example, inflammatory airways disease (chronic obstructive pulmonary disease) would be effectively treated by the compounds of this invention by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Further, the compounds of this invention can additionally be used to treat asthma, inflammatory bowel disease, rhinitis, pancreatitis, cystitis (interstitial cystitis), uveitis, inflammatory skin disorders, rheumatoid arthritis and edema resulting from trauma associated with burns, sprains or fracture by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used subsequent to surgical intervention (e.g. as post-operative analgesics) and to treat inflammatory pain of varied origins (e.g. osteoarthritis, rheumatoid arthritis, rheumatic disease, teno-synovitis and gout) as well as for the treatment of pain associated with angina, menstruation or cancer by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat diabetic vasculopathy, post capillary resistance or diabetic symptoms associated with insulitis (e.g. hyperglycemia, diuresis, proteinuria and increased nitrite and kallikrein urinary excretion) by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat inflammatory skin disorders such as psoriasis and eczema by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used as smooth muscle relaxants for the treatment of spasm of the gastrointestinal tract or uterus or in the therapy of Crohn's disease, ulcerative colitis or pancreatitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Such compounds may be used therapeutically to treat hyperreactive airways and to treat inflammatory events associated with airways disease e.g. asthma, and to control, restrict or reverse airways hyperreactivity in asthma by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat intrinsic and extrinsic asthma including allergic asthma (atopic or non-atopic) as well as exercise-induced bronchoconstriction, occupational asthma, viral or bacterial exacerbated asthma, other non-allergic asthmas and “wheezy-infant syndrome” by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may also be effective against pneumoconiosis, including aluminosis, anthracosis, asbestosis, chalicosis, ptilosis, siderosis, silicosis, tabacosis and byssinosis was well as adult respiratory distress syndrome, chronic obstructive pulmonary or airways disease, bronchitis, allergic rhinitis, and vasomotor rhinitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Additionally, they may be effective against liver disease, multiple sclerosis, atherosclerosis, Alzheimer's disease, septic shock e.g. as anti-hypovolemic and/or anti-hypotensive agents, cerebral edema, headache including cluster headache, migraine including prophylactic and acute use, closed head trauma, irritable bowel syndrome and nephritis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I, II and III. When a compound of Formula I, II and III is used contemporaneously with one or more other drugs, a pharmaceutical composition containing such other drugs in addition to the compound of Formula I, II and III is preferred. Accordingly, the pharmaceutical compositions of the present invention include those that also contain one or more other active ingredients, in addition to a compound of Formula I, II and III. Examples of other active ingredients that may be combined with a compound of Formula I, II and III, either administered separately or in the same pharmaceutical compositions, include, but are not limited to:
(1) morphine and other opiate receptor agonists including propoxyphene (Darvon); (2) non-steroidal antiinflammatory drugs (NSAIDs) including COX-2 inhibitors such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and the coxibs (celecoxib, valecoxib, rofecoxib and etoricoxib); (3) corticosteroids such as betamethasone, budesonide, cortisone, dexamethasone, hydrocortisone, methylprednisolone, prednisolone, prednisone and triamcinolone; (4) histamine H1 receptor antagonists such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, desloratadine, fexofenadine and levocetirizine; (5) histamine H2 receptor antagonists such as cimetidine, famotidine and ranitidine; (6) proton pump inhibitors such as omeprazole, pantoprazole and esomeprazole; (7) leukotriene antagonists and 5-lipoxygenase inhibitors such as zafirlukast, montelukast, pranlukast and zileuton; (8) drugs used for angina, myocardial ischemia including nitrates such as nitroglycerin and isosorbide nitrates, beta blockers such as atenolol, metoprolol, propranolol, acebutolol, betaxolol, bisoprolol, carteolol, labetalol, nadolol, oxprenolol, penbutolol, pindolol, sotalol and timolol, and calcium channel blockers such as diltiazam, verapamil, nifedipine, bepridil, felodipine, flunarizine, isradipine, nicardipine and nimodipine; (9) incontinence medications such as antimuscarinics, e.g., tolterodine and oxybutinin); (10) gastrointestinal antispasmodics (such as atropine, scopolamine, dicyclomine, antimuscarinics, as well as diphenoxylate); skeletal muscle relaxants (cyclobenzaprine, carisoprodol, chlorphenesin, chlorzoxazone, metaxalone, methocarbamol, baclofen, dantrolene, diazepam, or orphenadrine); (11) gout medications such as allopurinol, probenicid and colchicine; (12) drugs for rheumatoid arthritis such as methotrexate, auranofin, aurothioglucose and gold sodium thiomalate; (13) drugs for osteoporosis such as alendronate and raloxifene; decongestants such as pseudoephedrine and phenylpropanolamine; (14) local anesthetics; (15) anti-herpes drugs such as acyclovir, valacyclovir and famcyclovir; and (15) anti-emetics such as ondansetron and granisetron.
   Biological Evaluation

Assessing the Affinity of Selected Compounds to Bind to the Bradykinin B1 or B2 Receptor Radioligand binding assays are performed using membranes from CHO cells that stably express the human, rabbit, rat, or dog B1 receptors or CHO cells that express the human B2 receptor. For all receptor types, cells are harvested from culture flasks in PBS/1 mM EDTA and centrifuged at 1000×g for 10 minutes. The cell pellets are homogenized with a polytron in ice cold 20 mM HEPES, 1 mM EDTA, pH 7.4 (lysis buffer) and centrifuged at 20,000×g for 20 minutes. The membrane pellets are rehomogenized in lysis buffer, centrifuged again at 20,000×g and the final pellets are resuspended at 5 mg protein/ml in assay buffer (120 mM NaCl, 5 mM KCl, 20 mM HEPES, pH 7.4) supplemented with 1% BSA and frozen at −80° C.

On the day of assay, membranes are centrifuged at 14,000×g for 5 minutes and resuspended to the desired protein concentration in assay buffer containing 100 nM enaliprilat, 140 μg/mL bacitracin and 0.1% BSA. 3H-des-arg10, leu9 kallidin is the radioligand used for the human and rabbit B1 receptors, 3H-des-arg10 kallidin is used for the rat and dog B1 receptors, and 3H-bradykinin is used to label the human B2 receptor.

For all assays, compounds are diluted from DMSO stock solutions with 4 μL added to assay tubes for a final DMSO concentration of 2%. This is followed by the addition of 100 μL radioligand and 100 μL of the membrane suspension. Nonspecific binding for the B1 receptor binding assays is determined using 1 μM des-arg10 kallidin and nonspecific binding for the B2 receptor is determined with 1 μM bradykinin. Tubes are incubated at room temperature (22° C.) for 60 minutes followed by filtration using a Tomtec 96-well harvesting system. Radioactivity retained by the filter is counted using a Wallac Beta-plate scintillation counter.

The compounds of this invention have affinity for the B1 receptor in the above assay as demonstrated by results of less than 5 μM. It is advantageous that the assay results be less than 1 μM, even more advantageous for the results be less than 0.5 μM. It is further advantageous that compounds of this invention have affinity for the bradykinin B1 receptor over the bradykinin B2 receptor; more advantageously, the affinity for the B1 receptor is at least 10 fold, and preferably over 100 fold, over that for the B2 receptor.

Assay for Bradykinin B1 Antagonists

B1 agonist-induced calcium mobilization was monitored using a Fluorescence Imaging Plate Reader (FLIPR). CHO cells expressing the B1 receptor were plated in 96 or 384 well plates and allowed to incubate in Iscove's modified DMEM overnight. Wells were washed two times with a physiological buffered salt solution and then incubated with 4 uM Fluo-3 for one hour at 37° C. The plates were then washed two times with buffered salt solution and 100 uL of buffer was added to each well. Plates were placed in the FLIPR unit and allowed to equilibrate for two minutes. The test compound was then added in 50 ul volumes followed five minutes later by 50 ul of agonist (des-arg¹⁰ kallidin). Relative fluorescence peak heights in the absence and presence of antagonist were used to calculate the degree of inhibition of the B1 receptor agonist response by the test compound. Eight to ten concentrations of test compound were typically evaluated to construct an inhibition curve and determine IC50, values using a four-parameter nonlinear regression curve fitting routine.

Assay for Bradykinin Inverse Agonists

Inverse agonist activity at the human B1 receptor was evaluated using transiently transfected HEK293 cells. One day following transfection cell flasks were labeled overnight with 6 uCi/ml [³H]myo-inositol. On the day of assay, the media was removed and the attached cells were gently rinsed with 2×20 ml of phosphate-buffered saline. Assay buffer (HEPES buffered physiological salts, pH 7.4) was added and the cells were detached by tapping of the flask. The cells were centrifuged at 800×g for five minutes and resuspended at 1×10⁶ cells/ml in assay buffer supplemented with 10 mM lithium chloride. After 10 minutes at room temperature, one-half ml aliquots were distributed to tubes containing test compound or vehicle. After an additional 10 minutes the tubes were transferred to a 37° C. water bath for 30 minutes. The incubation was terminated by the addition of a 12% perchloric acid solution and the tubes were placed on ice for 30 minutes. The acid was then neutralized with KOH and the tubes centrifuged to pellet precipitated material. [³H]Inositol monophosphate formed was recovered by standard ion exchange chromatographic techniques and quantitated by liquid scintillation counting. Inverse agonist activity was determined by the degree to which a test compound reduced basal (cells incubated with vehicle) levels of [³H]inositol monophosphate accumulation.

Abbreviations Used

AIBN 2,2′-azobisisobutyronitrile
Bu butyl
DMF dimethylformamide
DMSO Dimethyl dimethyl sulfoxide
EDC or EDCI 1-(3-dimethylaminopropyl)-3-ethylcarbodiimide HCl
ES (or ESI)-MS electron spray ionization—mass spectroscopy
EtOAc ethyl acetate
HBT or HOBt 1-hydroxybenzotriazole hydrate
HPLC high pressure liquid chromatography
Me methyl
MeOH methanol
NBS N-bromosuccinimde
NMR nuclear magnetic resonance
Ph phenyl
rt room temperature
TEA triethylamine
Tf triflate (trifluoromethanesulfonyl)
TFA trifluoroacetic acid
THF tetrahydrofuran

The compounds of the present invention can be prepared according to the following reaction schemes and examples, or modifications thereof, using readily available starting materials, reagents, and conventional synthesis procedures. In these reactions, it is also possible to make use of variants which are themselves known to those of ordinary skill in this art, but are not mentioned in greater detail.

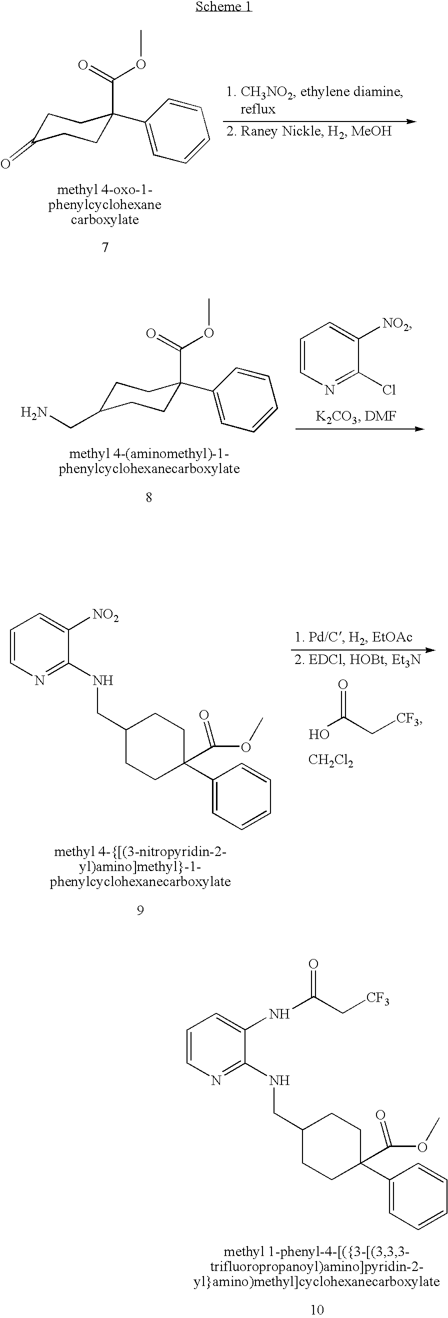

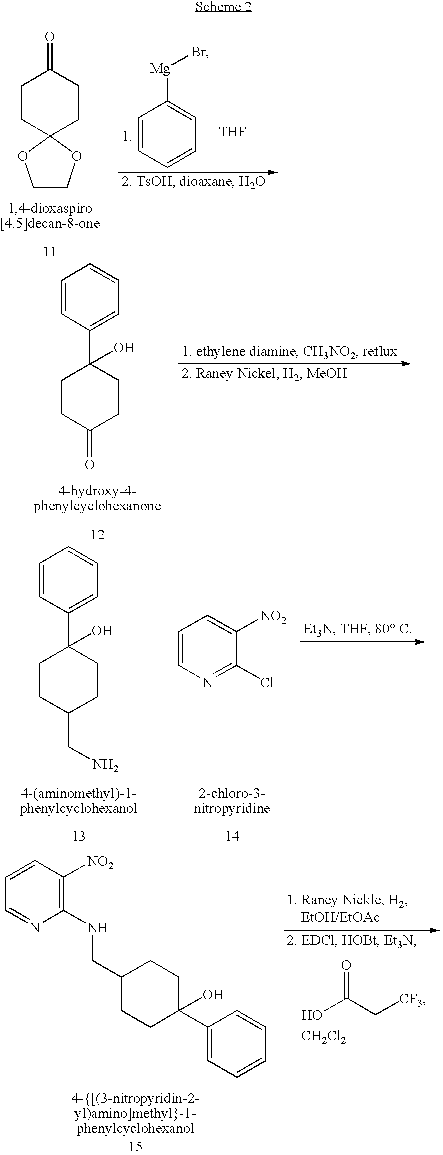

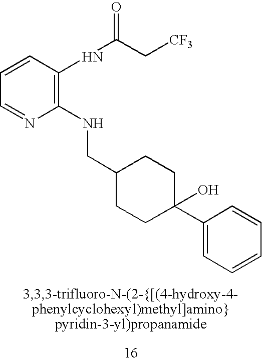

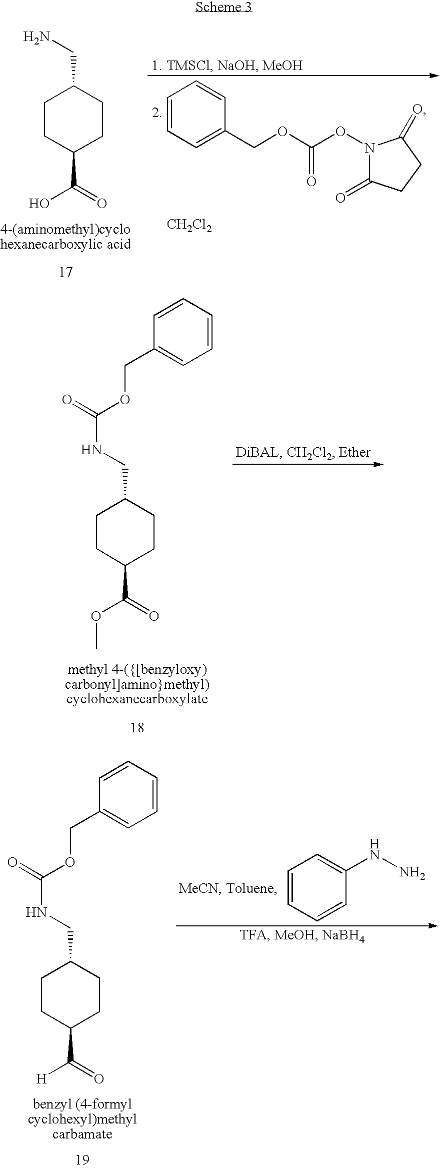

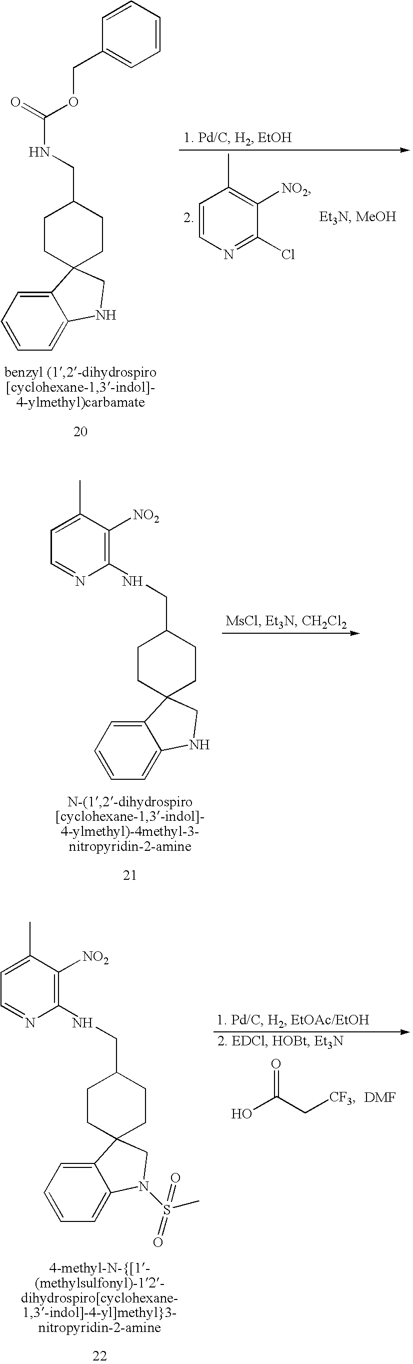

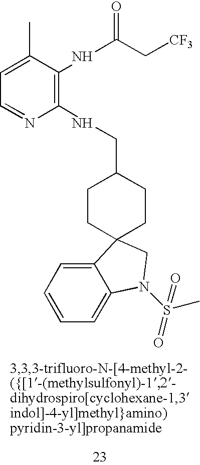

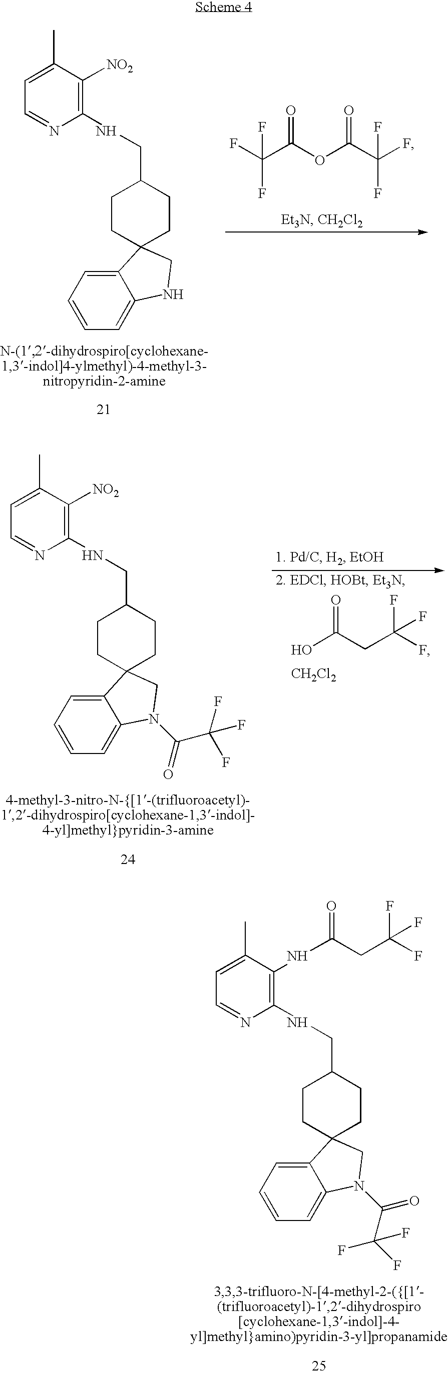

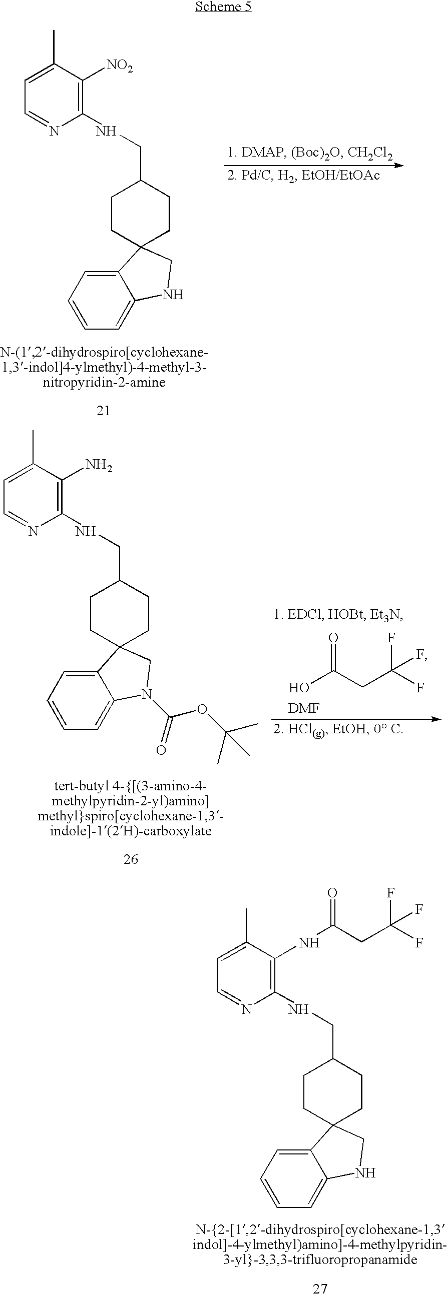

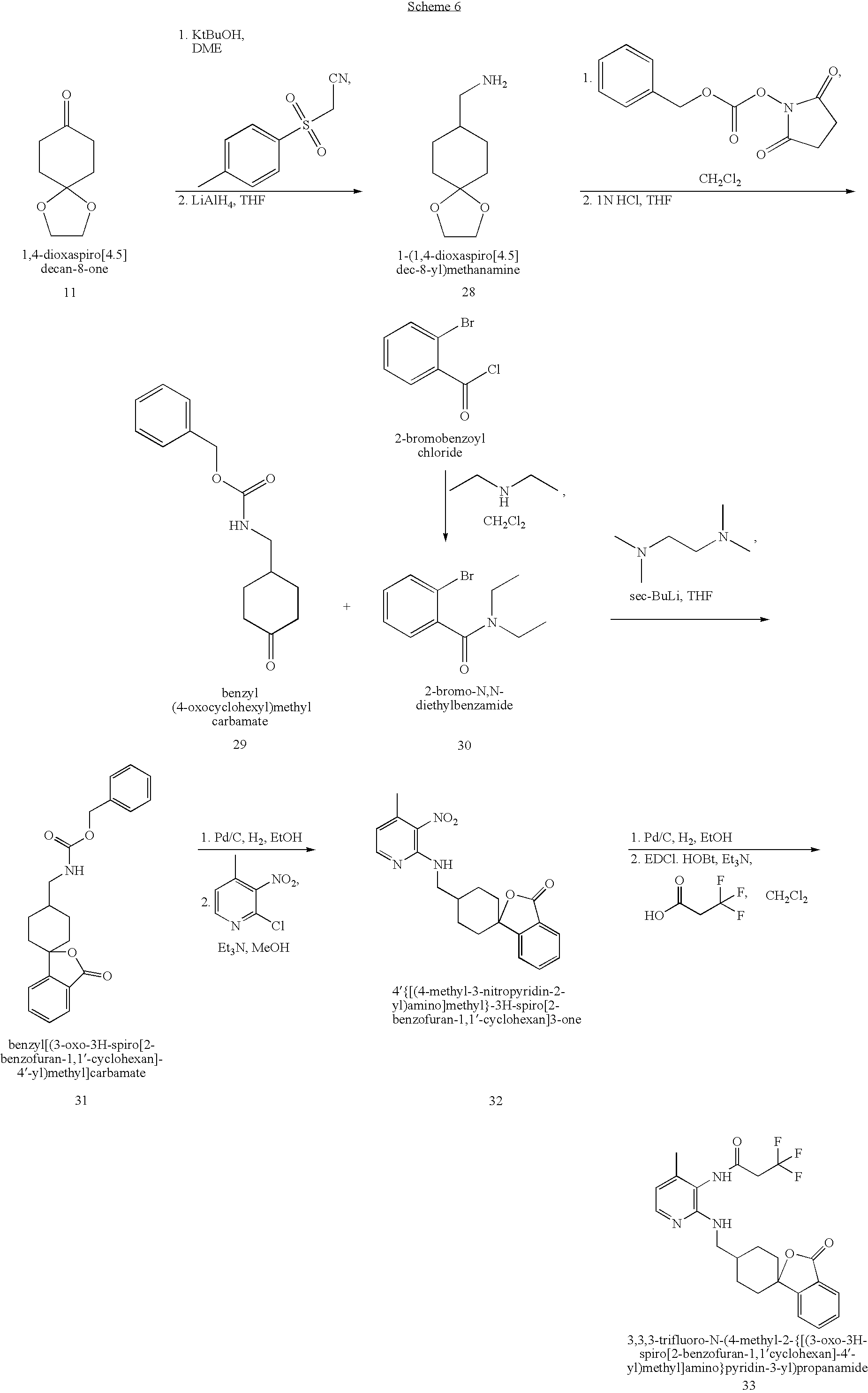

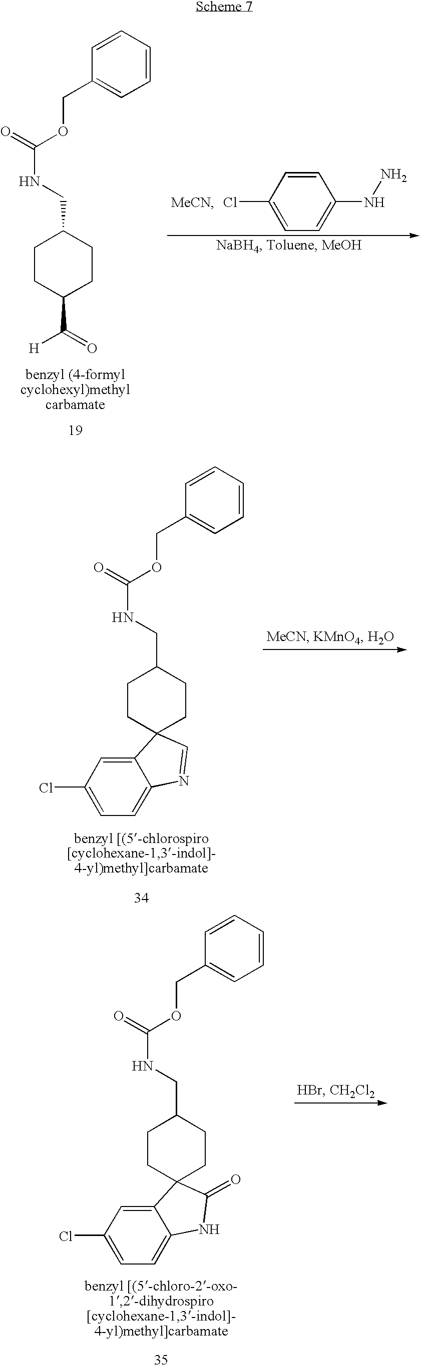

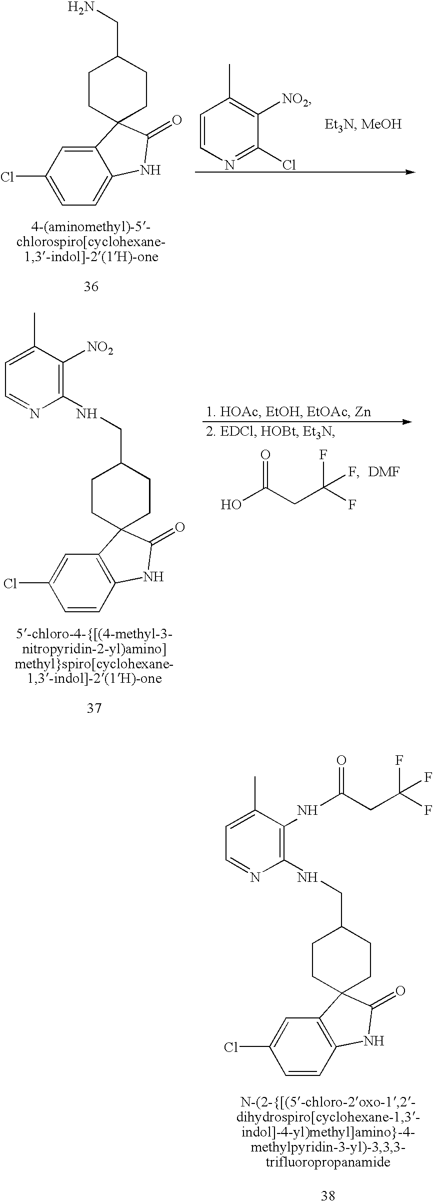

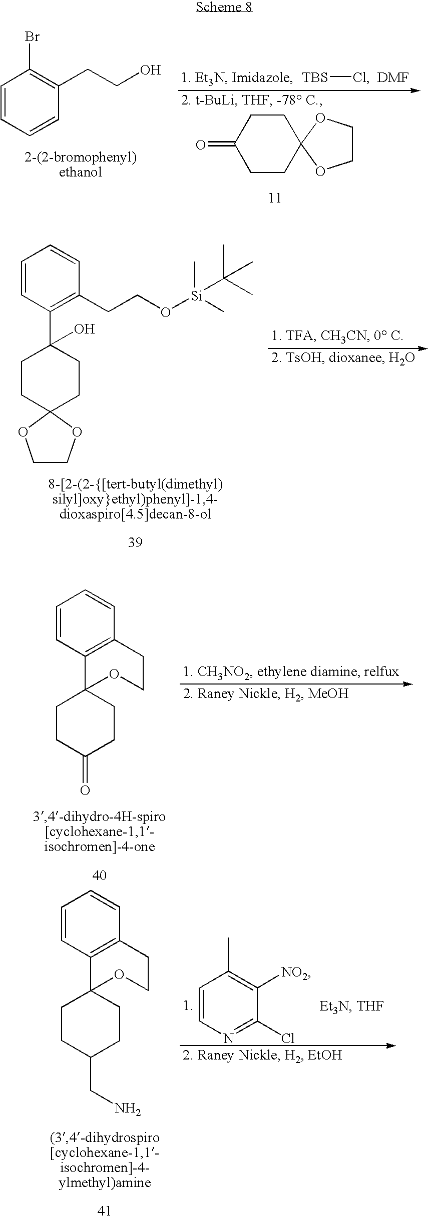

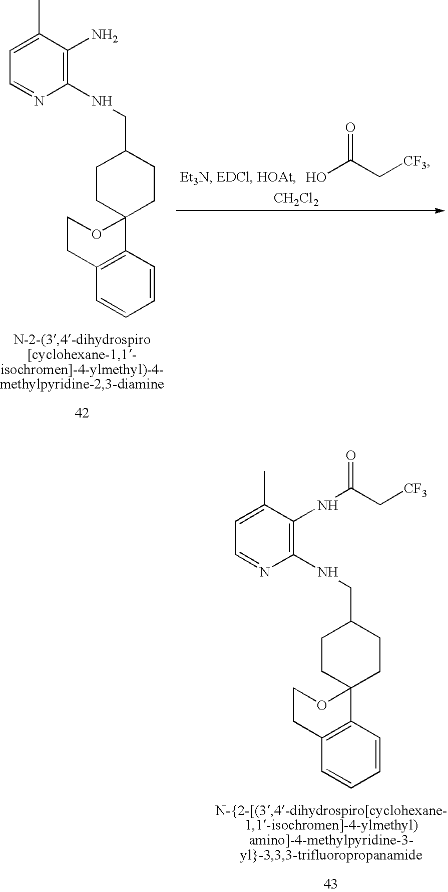

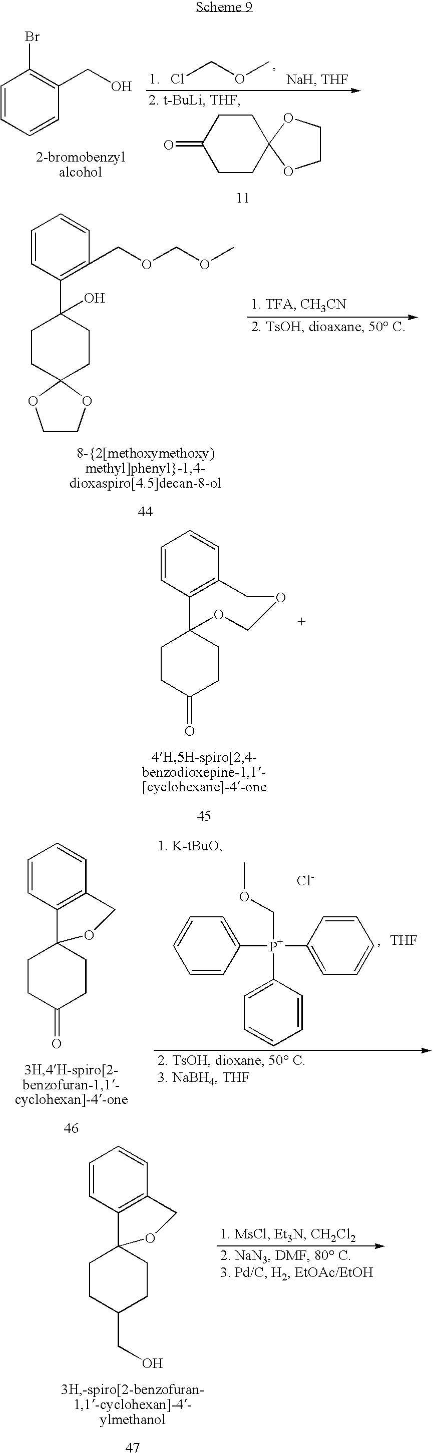

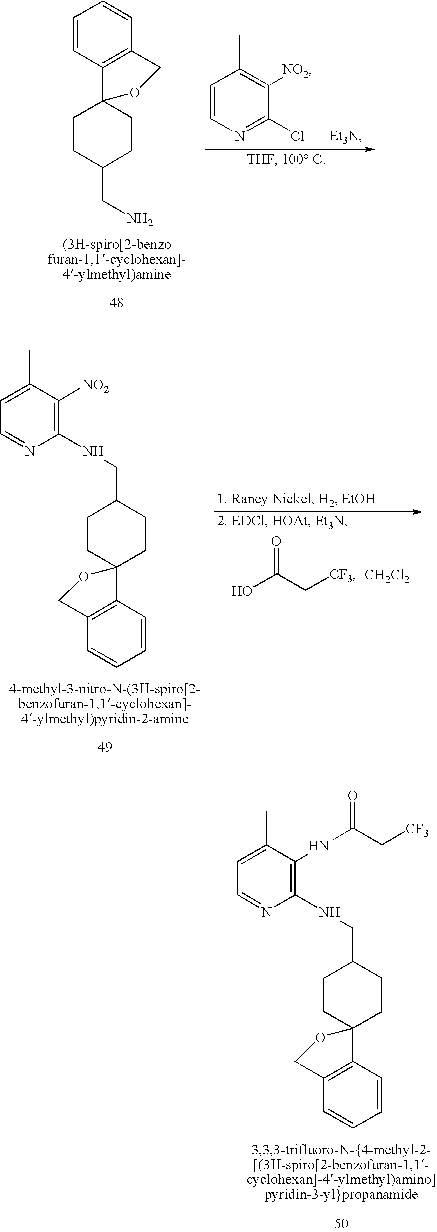

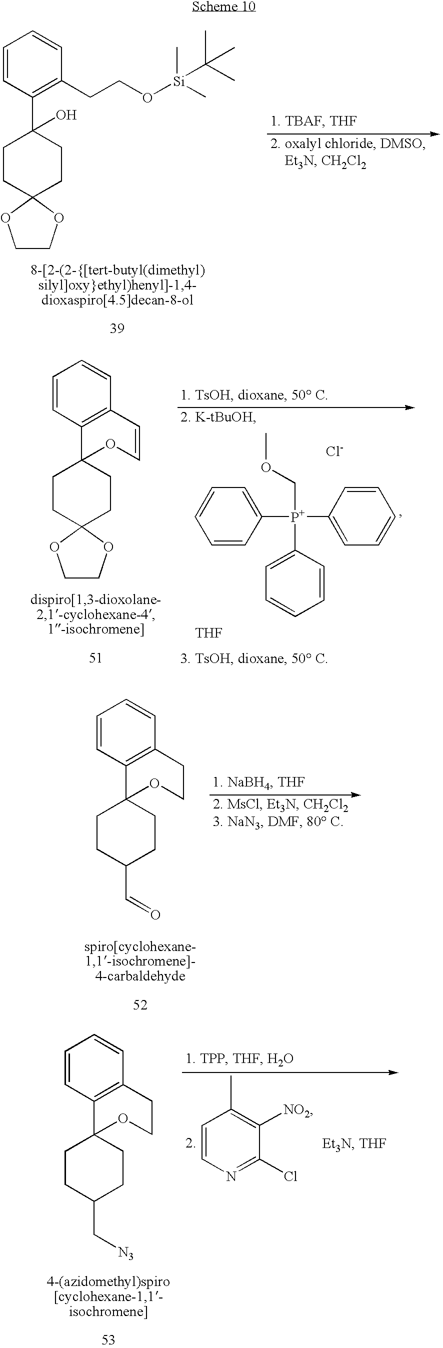

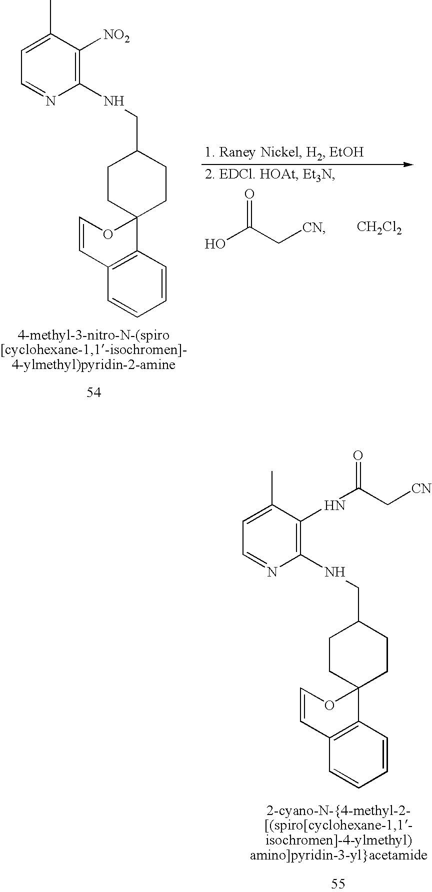

### Experimental for the Preparation of Compound 10 (Example 1 in Table B

### methyl 1-phenyl-4-[({3-[(3,3,3-trifluoropropanoyl)amino]pyridin-2-yl}amino)methyl]cyclohexanecarboxylate

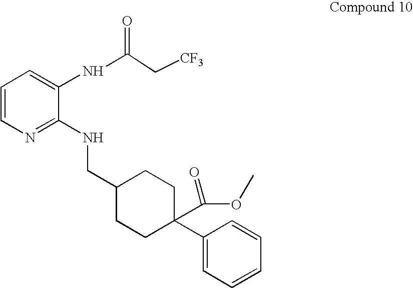

A solution of methyl 4-oxo-1-phenylcyclohexanecarboxylate, 7 (481 mg, 2.07 mmol) in nitromethane (10 mL) was heated to reflux. Then ethylene diamine (15 uL, 0.22 mmol) was added and an additional 1 mL of nitromethane was added. Upon completion, the reaction mixture was cooled to room temperature and concentrated in vacuo. Silica gel chromatography (17%-25% EtOAc/hexanes) gave methyl 4-(nitromethylene)-1-phenylcyclohexanecarboxylate.

To a solution of methyl 4-(nitromethylene)-1-phenylcyclohexanecarboxylate (356 mg, 1.29 mmol) in MeOH (5 mL) was added Raney nickel (1 mL of a slurry in water). The resulting slurry was flushed with nitrogen and a hydrogen balloon was placed over the reaction. After approximately one hour, the reaction mixture was filtered through a pad of celite, washing with 200 mL of EtOH/MeOH (1:1), and concentrated in vacuo. Silica gel chromatography (2%-5% MeOH/CH₂Cl₂ saturated with NH₃) gave methyl 4-(aminomethyl)-1-phenylcyclohexanecarboxylate, 8.

To a solution of 8 (165 mg, 0.67 mmol) in DMF (2.5 mL), was added 2-chloro-3-nitropyridine (116 mg, 0.73 mmol). The resulting solution was heated to 80° C. and stirred overnight. The reaction mixture was cooled, diluted with EtOAc (150 mL), and washed with water (4×25 mL) and brine (1×25 mL). The organic phase was dried over magnesium sulfate, filtered, and concentrated to give methyl 4-{[(3-nitropyridin-2-yl)amino]methyl}-1-phenylcyclohexanecarboxylate, 9.

To a solution of 9 (261 mg, 0.67 mmol) in EtOAc (5 mL) was added Pd/C (100 mg, 10% by weight). The resulting slurry was flushed with nitrogen and a hydrogen balloon was placed over the reaction. After two hours, an additional 100 mg of Pd/C and 4 mL of MeOH were added. Upon completion, the reaction mixture was filtered through a pad of celite and concentrated. Silica gel chromatography (1% MeOH/CH₂Cl₂ saturated with NH₃) gave methyl 4-{[(3-aminopyridin-2-yl)amino]methyl}-1-phenylcyclohexanecarboxylate.

To a solution of methyl 4-{[(3-aminopyridin-2-yl)amino]methyl}-1-phenylcyclohexanecarboxylate (185 mg, 0.54 mmol) in DMF (2.5 mL), was added 3,3,3,-trifluoropropionic acid (0.07 mL, 0.817), 1-[3-(dimethylamino)propyl]-3-ethylcarbodiimide hydrochloride (158 mg, 0.82 mmol), 1-hydroxybenzotriazole hydrate (110 mg, 0.82 mmol), and Et₃N (0.18 mL, 1.3 mmol). The resulting solution stirred for two days and was diluted with EtOAc (150 mL), washed with water (4×50 mL), and brine (1×25 mL). The organic phase was dried over magnesium sulfate, filtered, and concentrated. Silica gel chromatography (1%-2% MeOH/CH₂Cl₂ saturated with NH₃) gave methyl 1-phenyl-4-[({3-[(3,3,3-trifluoropropanoyl)amino]pyridin-2-yl}amino)methyl]cyclohexanecarboxylate, 10, as a mix of isomers (˜1:1). LCMS (ES) m/z 450.1 (M+H)⁺.

### Experimental for the Preparation of Compound 16 Example 8 in Table C

### 3,3,3-trifluoro-N-(2-{[(4-hydroxy-4-phenylcyclohexyl)methyl]amino}pyridin-3-yl)propanamide

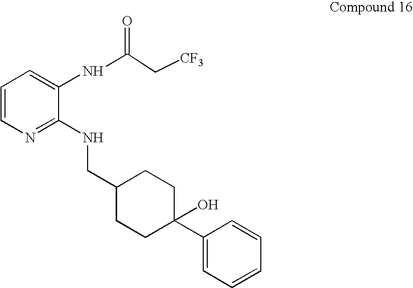

To a solution of phenylmagnesium bromide (8.71 g, 48.02 mmol) in THF (100 mL) at −78° C., was added 1,4-dioxaspiro[4.5]decan-8-one, 11 (5.00 g, 32.01 mmol) in THF (40 mL). The reaction mixture was slowly warmed to room temperature. Upon completion, the reaction was quenched with water and concentrated in vacuo. The crude residue was diluted with EtOAc (300 mL) and washed with water (1×50 mL) and brine (1×50 mL). The organic phase was dried over sodium sulfate, filtered, and concentrated to give 8-phenyl-1,4-dioxaspiro[4.5]decan-8-ol, which was used without further purification.

A solution of 8-phenyl-1,4-dioxaspiro[4.5]decan-8-ol (7.50 g, 32.01 mmol) and p-toluenesulfonic acid monohydrate (9.13 g, 48.02 mmol) in 200 mL of dioxane/water (9:1) was heated to 50° C. Upon completion, the reaction mixture was cooled, poured into EtOAc (300 mL), washed with water (1×100 mL), and brine (1×100 mL). The organic phase was dried over magnesium sulfate, filtered, and concentrated. Silica gel chromatography (25%-50% EtOAc/hexanes) gave 4-hydroxy-4-phenylcyclohexanone, 12.

To a solution of 12 (5.00 g, 26.28 mmol) in nitromethane (100 mL), ethylene diamine (0.18 mL, 2.63 mmol) was added, and the resulting solution heated to reflux. After four hours, the reaction mixture was cooled and concentrated in vacuo. Silica gel chromatography (25% EtOAc/hexanes) gave 4-(nitromethyl)-1-phenylcyclohex-3-en-1-ol.

A solution of 4-(nitromethyl)-1-phenylcyclohex-3-en-1-ol (4.15 g, 17.79 mmol) in MeOH (100 mL) was flushed with nitrogen before Raney nickel (˜10 mL of a slurry in water) was added. A hydrogen balloon was placed over the reaction, and it stirred at room temperature for approximately five hours. The reaction mixture was filtered through a pad of celite and concentrated. Silica gel chromatography (5%-10% MeOH/CH₂Cl₂ with 1% NH₄OH) gave 4-(aminomethyl)-1-phenylcyclohexanol, 13.

A solution of 2-chloro-3-nitropyridine, 14 (488 mg, 3.08 mmol), intermediate 13 (527 mg, 2.57 mmol), and Et₃N (1.43 mL, 10.27 mmol) in THF (5 mL) in a sealed flask was heated to 80° C. Upon completion, the reaction mixture was cooled and concentrated in vacuo. Silica gel chromatography (0%-20% Ether/CH₂Cl₂) gave 4-{[(3-nitropyridin-2-yl)amino]methyl}-1-phenylcyclohexanol, 15, as a mixture of isomers. LCMS (ES) m/z 328.3 (M+H)⁺.

A solution of 15 (480 mg, 1.47 mmol) in 10 mL EtOH/EtOAc (1:1) was flushed with nitrogen before Raney nickel (˜2 mL of a slurry in water) was added. A hydrogen balloon was placed over the reaction and it stirred at room temperature for approximately two hours. The reaction mixture was filtered through a pad of celite and concentrated to give 4-{[(3-nitropyridin-2-yl)amino]methyl}-1-phenylcyclohexanol, which was used without further purification. LCMS (ES) m/z 298.4 (M+H)⁺.

To a solution of 4-{[(3-nitropyridin-2-yl)amino]methyl}-1-phenylcyclohexanol (225 mg, 0.76 mmol) in CH₂Cl₂ (3 mL), Et₃N (0.53 mL, 3.79 mmol), 1-{3-(dimethylamino)propyl]-3-ethylcarbodiimde hydrochloride (435 mg, 2.27 mmol), 1-hydroxybenzotriazole hydrate (307 mg, 2.27 mmol), and 3,3,3-trifluoropropionic acid (0.12 mL, 0.91 mmol) were added. After overnight stirring, the reaction mixture was poured into EtOAc (50 mL) and washed with water (1×20 mL) and brine (1×20 mL). The organic phase was dried over sodium sulfate, filtered, and concentrated. The crude product was purified via silica gel chromatography (50%-75% EtOAc/hexanes). Further purification using reverse phase HPLC gave 3,3,3-trifluoro-N-(2-{[(4-hydroxy-4-phenylcyclohexyl)methyl]amino}pyridin-3-yl)propanamide, 16. LCMS (ES) m/z 408.3 (M+H)⁺.

### Experimental for the Preparation of Compound 23 Example 2 in Table C

### 3,3,3-trifluoro-N-[4-methyl-2-({[1′-(methylsulfonyl)-1′,2′-dihydrospiro[cyclohexane-1,3′-indol]4-yl]methyl}amino)pyridin-3-yl]propanamide

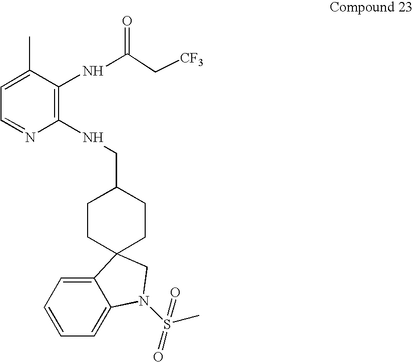

4-(aminomethyl)cyclohexanecarboxylic acid, 17 (10.60 g, 67.43 mmol) was suspended in MeOH (130 mL) and chlorotrimethylsilane (10 mL, 1.17 mmol) was added. The resulting solution was heated to 60° C. After approximately four hours, the reaction mixture was cooled and concentrated. To this residue was added sodium hydroxide (3.15 g, 1.17 mmol) in MeOH (100 mL). Upon completion, the reaction mixture was concentrated and azeotroped with EtOAc (150 mL). The crude residue was taken up in CH₂Cl₂ (300 mL) and filtered. Concentration of the filtrate gave methyl 4-(aminomethyl) cyclohexanecarboxylate. LCMS (ES) m/z 172.3 (M+H)⁺.

To a solution of methyl 4-(aminomethyl)cyclohexanecarboxylate (4.99 g, 29.12 mmol) in CH₂Cl₂ (30 mL) at 0° C., was added N-(benzyloxycarbonyloxy)succinimide (6.91 g, 27.74 mmol). The reaction mixture was warmed to room temperature and stirred overnight. Then the reaction mixture was loaded directly onto a silica gel column (1%-5% MeOH/CH₂Cl₂) to give methyl 4-({[(benzyloxy)carbonyl]amino}methyl)cyclohexanecarboxylate, 18. LCMS (ES) m/z 306.2 (M+H)⁺.

A solution of 18 (5.83 g, 19.09 mmol) in ether (190 ml) was cooled to −78° C. CH₂Cl₂ (60 mL) was added to help the solubility before diisobutylaluminum hydride (21.19 mL, 1M in CH₂Cl₂) was added dropwise. The reaction stirred for approximately five minutes, and then 3.1 mL of water was introduced via syringe. The dry ice bath was then removed, and the reaction stirred overnight. The reaction mixture was filtered through a pad of celite and concentrated in vacuo. Silica gel purification (10%-40% EtOAc/hexanes) gave benzyl (4-formylcyclohexyl)methylcarbamate, 19. LCMS (ES) m/z 276.2 (M+H)⁺.

To a solution of 19 (2.17 g, 7.87 mmol) in toluene (80 mL), was added CH₃CN (8 mL). The resulting solution was cooled to 0° C. and phenylhydrazine (0.93 mL, 9.44 mmol) was added. After approximately thirty minutes, the reaction mixture was transfer to a 10° C. bath. One hour later, trifluoroacetic acid (2.43 mL, 31.48 mmol) was added, and the reaction mixture was allowed to warm to room temperature. The reaction mixture was eventually heated to 50° C. to force the formation of the imine. After overnight stirring at 50° C., the reaction mixture was cooled to 0° C. and MeOH (8 mL) followed by sodium borohydride (253 mg, 6.69 mmol) were added. Additional sodium borohydride was added. Upon completion, the reaction mixture was washed with an aqueous 10% NH₄OH solution (1×11 mL), then with water (3×20 mL), and brine (2×20 mL). The organic phase was dried over sodium sulfate, filtered, and concentrated. Silica gel chromatography (1%-5% MeOH/CH₂Cl₂) gave benzyl (1′,2′-dihydrospiro[cyclohexane-1,3′-indol]4-ylmethyl)carbamate, 20, as a mix of isomers. LCMS (ES) m/z 351.3 (M+H)⁺.

A solution of 20 (1.83 g, 5.22 mmol) in EtOH (50 mL) was flushed with nitrogen before Pd/C (550 mg, 10% by weight) was added. A hydrogen balloon was placed over the reaction. Upon completion, the reaction mixture was filtered through a pad of celite, and concentrated to give (1′,2′-dihydrospiro[cyclohexane-1,3′-indol]4-ylmethyl)amine, which was used without further purification. LCMS (ES) m/z 217.3 (M+H)⁺.

To a solution of (1′,2′-dihydrospiro[cyclohexane-1,3′-indol]4-ylmethyl)amine (1.11 g, 5.18 mmol) in MeOH (10 mL) were added Et₃N (0.87 mL, 6.21 mmol) followed by 2-chloro-4-methyl-3-nitropyridine (938 mg, 5.44 mmol). The reaction was heated to reflux and left to stir for several days. Upon completion, the major isomer crystalized out of the reaction mixture and was filtered, washing with MeOH to give N-(1′,2′-dihydrospiro[cyclohexane-1,3′-indol]-4-ylmethyl)-4methyl-3-nitropyridin-2-amine, 21. LCMS (ES) m/z 353.2 (M+H)⁺.

To a solution of 21 (200 mg, 0.57 mmol) in CH₂Cl₂ (5 mL) at 0° C., was added Et₃N (0.10 mL, 0.74 mmol) and mesyl chloride (0.06 mL, 0.74 mmol). Upon completion, the reaction mixture was loaded directly onto a silica gel column (1-15% MeOH/CH₂Cl₂) to give 4-methyl-N-{[1′-(methylsulfonyl)-1′,2′dihydrospiro[cyclohexane-1,3′-indol]4-yl]methyl}-3-nitropyridin-2-amine, 22. LCMS (ES) m/z 431.2 (M+H)⁺.

A solution of 22 (249 mg, 0.58 mmol) in 10 mL of EtOAc/EtOH (1:1) was flushed with nitrogen before Pd/C (50 mg, 10% by weight) was added. A hydrogen balloon was placed over the reaction. Upon completion, the reaction mixture was filtered through a pad of celite washing with EtOH, and concentrated. Silica gel chromatography (1%-15% MeOH/CH₂Cl₂) gave 4-methyl-N²-{[1′-(methylsulfonyl)-1′,2′-dihydrospiro[cyclohexane-1,3′-indol]-4-yl]methyl}pyridine-2,3-diamine. LCMS (ES) m/z 401.3 (M+H)⁺.

To a solution of 4-methyl-N²-{[1′-(methylsulfonyl)-1′,2′-dihydrospiro[cyclohexane-1,3′-indol]-4-yl]methyl}pyridine-2,3-diamine (102 mg, 0.26 mmol) and 1-hydroxybenzotriazole hydrate (8 mg, 0.05 mmol) in DMF (2 mL), was added 3,3,3-trifluoropropionic acid (0.03 mL, 0.31 mmol), Et₃N (0.05 mL, 0.36 mmol), and 1-[3-(dimethylamino)propyl]ethyl carbodiimide hydrochloride (68 mg, 0.36 mmol). After overnight stirring, the reaction mixture was partitioned between water (30 mL) and EtOAc (40 mL). The organic phase was washed with saturated CuSO₄ (1×20 ml), half brine (2×20 mL), and brine (1×20 mL), then dried over sodium sulfate, filtered, and concentrated. The product was titrated with CH₂Cl₂ and dried to give 3,3,3-trifluoro-N-[4-methyl-2-({[1′-(methylsulfonyl)-1′,2′-dihydrospiro[cyclohexane-1,3′-indol]-4-yl]methyl}amino)pyridin-3-yl]propanamide, 23. LCMS (ES) m/z 511.3 (M+H)⁺.

### Experimental for the Preparation of Compound 25 Example 6 in Table C

### 3,3,3-trifluoro-N-[4-methyl-2-({[1′-(trifluoroacetyl)-1′,2′-dihydrospiro[cyclohexane-1,3′-indol]-4-yl]methyl}amino)pyridin-3-yl]propanamide

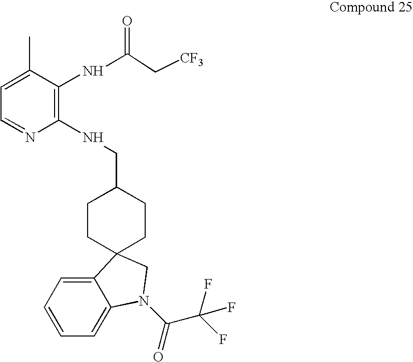

To a solution of 21 (200 mg, 0.57 mmol) in CH₂Cl₂ (5 mL), was added trifluoroacetic anhydride (0.10 mL, 0.74 mmol) followed by Et₃N (0.10 mL, 0.74). After approximately thirty minutes, the reaction mixture was loaded directly onto a silica gel column (0%-4% MeOH/CH₂Cl₂) to give 4-methyl-3-nitro-N-{[1′-(trifluoroacetyl)-1′,2′-dihydrospiro[cyclohexane-1,3′-indol]-4-yl]methyl}pyridin-2-amine, 24. LCMS (ES) m/z 449.3 (M+H)⁺.

To a suspension of 24 (234 mg, 0.52 mmol) in EtOH (6 mL), EtOAc (4 mL) was added to improve solubility. The resulting solution was evacuated and filled with nitrogen three times before Pd/C (30 mg, 10% by weight) was added. The reaction mixture stirred under hydrogen balloon for approximately six hours before it was filtered through a pad of celite, washing with 50 mL of MeOH with 1% NH₄OH. Concentration of the filtrate gave 4-methyl-N˜2˜-{[1′-(trifluoroacetyl)-1′,2′-dihydrospiro[cyclohexane-1,3′-indol]-4-yl]methyl}pyridine-2,3-diamine, which was used without further purification. LCMS (ES) m/z 419.3 (M+H)⁺.

To a solution of 4-methyl-N˜2˜-{[1′-(trifluoroacetyl)-1′,2′-dihydrospiro[cyclohexane-1,3′-indol]4-yl]methyl}pyridine-2,3-diamine (109 mg, 0.26 mmol) in DMF (2 mL), was added 1-hydroxybenzotriazole hydrate (8.0 mg, 0.05 mmol), trifluoropropionic acid (0.03 mL, 0.31 mmol), 1-[3-(dimethylamino)propyl]-3-ethylcarbodiimide hydrochloride (70.1 mg, 0.37 mmol), and Et₃N (0.054 mL, 0.39 mmol). The reaction stirred at room temperature for approximately four days. Additional trifluoropropionic acid, EDCI, and Et₃N were added as necessary to force the reaction to completion. Upon completion, the reaction mixture was concentrated in vacuo, and loaded directly onto a silica gel column (1-10% MeOH/CH₂Cl₂) to give 3,3,3-trifluoro-N-[4-methyl-2-({[1′-(trifluoroacetyl)-1′,2′-dihydrospiro[cyclohexane-1,3′-indol]-4-yl]methyl}amino)pyridin-3-yl]propanamide, 25. LCMS (ES) m/z 529.3 (M+H)⁺.

### Experimental for the Preparation of Compound 27 Example 10 in Table C

### N-{2-[(1′,2′-dihydrospiro[cyclohexane-1,3′-indol]4-ylmethyl)amino]4-methylpyridin-3-yl}-3,3,3-trifluoropropanamide

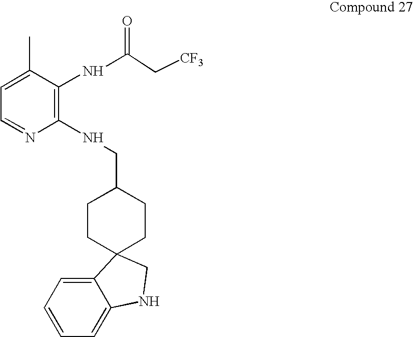

To a solution of 21 (100 mg, 0.28 mmol) and 4-(dimethylamino)pyridine (7.0 mg, 0.06 mmol) in CH₂Cl₂ (3 mL) at 0° C. was added (Boc)₂O (68 mg, 0.31 mmol). The reaction mixture was warmed to room temperature and stirred overnight. The reaction mixture was then purified by a silica gel flash column (0%-2% MeOH/CH₂Cl₂) to give tert-butyl 4-{[(4-methyl-3-nitropyridin-2-yl)amino]methyl}spiro[cyclohexane-1,3′-indole]-1′(2′H)-carboxylate. LCMS (ES) m/z 453.2 (M+H)⁺.

A solution of tert-butyl 4-{[(4-methyl-3-nitropyridin-2-yl)amino]methyl}spiro[cyclohexane-1,3′-indole]-1′(2′H)-carboxylate (100 mg, 0.22 mmol) in 6 mL of EtOH/EtOAc (1:1) was flushed with nitrogen before Pd/C (˜25 mg, 10% by weight) was added. The reaction mixture was again flushed with nitrogen. A hydrogen balloon was placed over the reaction. Upon completion, the reaction was flushed with nitrogen, filtered through a pad of celite, and concentrated to give tert-butyl 4-{[(3-amino-4-methylpyridin-2-yl)amino]methyl}spiro[cyclohexane-1,3′-indole]-1′(2′H)-carboxylate, 26, which was used without further purification.

To a solution of 26 (84 mg, 0.20 mmol) and 1-hydroxybenzotriazole hydrate (6 mg, 0.04 mmol) in DMF (1.4 mL), was added trifluoropropionic acid (0.02 mL, 0.24 mmol), Et₃N (0.04 mg, 0.28 mmol), and 1-[3-(dimethylamino)propyl]ethyl carbodiimide hydrochloride (53 mg, 0.28 mmol). After overnight stirring, the reaction mixture was partitioned between EtOAc and water. The organic phase was then washed with saturated CuSO₄ (1×), half brine (2×), and brine (1×), dried over sodium sulfate, filtered, and concentrated. The crude product was purified via silica gel chromatography (2%-17% MeOH/CH₂Cl₂) to give tert-butyl 4-[({4-methyl-3-[(3,3,3-trifluoropropanoyl) amino]pyridin-2-yl}amino)methyl]spiro[cyclohexane-1,3′-indole]-1′(2′H)-carboxylate. LCMS (ES) m/z 533.3 (M+H)⁺.

Tert-butyl 4-[({4-methyl-3-[(3,3,3-trifluoropropanoyl)amino]pyridin-2-yl}amino)methyl]spiro[cyclohexane-1,3′-indole]-1′(2′H)-carboxylate (65 mg, 0.12 mmol) was dissolved in EtOH (4 mL) and cooled to 0° C. with no stirring. HCl gas was bubbled through the reaction mixture for approximately three minutes. The reaction sat at 0° C. for approximately fifteen minutes before nitrogen gas was bubbled through the solution for approximately forty minutes. The EtOH was removed in vacuo to give N-{2-[(1′,2′-dihydrospiro[cyclohexane-1,3′-indol]4-ylmethyl)amino]4-methylpyridin-3-yl}-3,3,3-trifluoropropanamide, 27, as the HCl salt. LCMS (ES) m/z 433.2 (M+H)⁺.

### Experimental for the Preparation of Compound 33 Example 15 in Table C

### 3,3,3-trifluoro-N-(4-methyl-2-{[(3-oxo-3H-spiro[2-benzofuran-1,1′-cyclohexan]-4′-yl)methyl]amino}pyridin-3-yl)propanamide

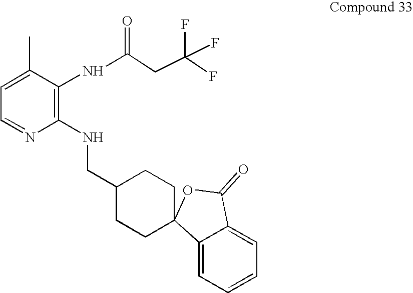

P-toluenesulfonylacetonitrile (6.25 g, 32.01 mmol) and 11 (5 g, 32.01 mmol) were dissolved in DME (160 mL) and EtOH (2 mL) and cooled to 0° C. Potassium t-butoxide (7.18 g, 64.03 mmol) was added. The reaction mixture stirred at 0° C. for thirty minutes and was warmed to room temperature. Upon completion, the reaction mixture was concentrated in vacuo. Water (40 mL) and NaCl were added to the residue, and a precipitate formed. After filtering through celite, the filtrate was extracted with Ether (5×50 mL). The combined organic phases were washed with brine, dried over sodium sulfate, filtered, and concentrated. Short-path distillation (˜105 to 115° C.) gave 1,4-dioxaspiro[4.5]decane-8-carbonitrile.

Lithium aluminum hydride (9.42 mL of a 1M solution in THF) was cooled to 0° C. 1,4-dioxaspiro[4.5]decane-8-carbonitrile (1.05 g, 6.28 mmol) dissolved in THF (4 mL) was then added over to the lithium aluminum hydride solution slowly via cannula. The reaction mixture was then heated to reflux for two hours and left to stand at room temperature overnight. The reaction was quenched with the sequential addition of water (0.36 mL), aqueous 15% NaOH (0.36 mL), and water (1.06 mL). The resulting suspension was filtered, washing the solids with THF (3×30 mL). Concentration of the filtrate gave 1-(1,4-dioxaspiro[4.5]dec-8-yl)methanamine, 28, which was used without further purification. LCMS (ES) m/z 172.3 (M+H)⁺.

To a solution of 28 (1.07 g, 6.28 mmol) in CH₂Cl₂ (60 mL), was added N-(benzyloxycarbonyloxy)succinimide (1.72 g, 6.91 mmol). The reaction mixture stirred at room temperature for approximately one hour before most of the solvent was removed (˜55 mL) in vacuo. The crude product was purified by a silica gel chromatography (8%-12% MeOH/CH₂Cl₂) to give benzyl 1,4-dioxaspiro[4.5]dec-8-ylmethylcarbamate. LCMS (ES) m/z 306.2 (M+H)⁺.

To a solution of benzyl 1,4-dioxaspiro[4.5]dec-8-ylmethylcarbamate (1.48 g, 4.86 mmol) in THF (48 mL) at 0° C. was added 1N HCl (13.11 mL). The ice bath was then removed, and the reaction mixture stirred overnight. Then, the reaction mixture was heated to 50° C. Upon completion, the reaction mixture was neutralized with 1N NaOH, THF was removed in vacuo, and the remaining water layer was extracted with EtOAc (2×50 mL). The combined organics were dried over sodium sulfate, filtered, and concentrated. Silica gel chromatography (1%-7% MeOH/CH₂Cl₂) gave benzyl (4-oxocyclohexyl)methylcarbamate, 29. LCMS (ES) m/z 262.2 (M+H)⁺.

To a solution of 2-bromobenzoyl chloride (5.36 mL, 41.01 mmol) in CH₂Cl₂ (57 mL) at 0° C., was added diethylamine dropwise. The reaction mixture stirred at 0° C. for approximately thirty minutes before it was diluted with CH₂Cl₂ (100 mL) and washed with water, aqueous 5% NaHCO₃, and brine. The organic phase was dried over sodium sulfate, filtered, and concentrated to give 2-bromo-N,N-diethylbenzamide, 30, which was used without further purification. LCMS (ES) m/z 256.1 (M+H)⁺.

Compound 29 (200 mg, 0.77 mmol) was placed in a flame dried flask and azeotroped with toluene (3×). In a separate flame dried flask was added dry THF (30 mL) and N,N,N′,N′-tetramethylethylenediamine (0.24 mL, 1.57 mmol). The resulting solution was cooled to −78° C. and sec-butyllithium (1.21 mL of a 1.3M solution in cyclohexane) was added. The benzamide, 29, was diluted with a small amount of THF and added over to the butyllithium solution via cannula. In a separate flame dried flask, 30 (271 mg, 1.53 mmol) in THF (5 mL) was cooled to −78° C. and added over to the reaction mixture. The reaction mixture was warmed to room temperature and stirred overnight. The next morning, the reaction was quenched with a few drops of water and concentrated in vacuo. The crude product was purified by reverse phase chromatography to give benzyl [(3-oxo-3H-spiro[2-benzofuran-1,1′-cyclohexan]-4′-yl)methyl]carbamate, 31.

A solution of 31 (90 mg, 0.25 mmol) in EtOH (2 mL) was evacuated with and filled with nitrogen three times. Pd/C (20 mg, 10% by weight) was then added, and the reaction vessel was again evacuated and filled with nitrogen three times. After a final evacuation, a hydrogen balloon was placed over the reaction, and it stirred overnight. The reaction mixture was filtered through a pad of celite, washing with methanol containing 1% NH₄OH, and concentrated to give 4′-(aminomethyl)-3H-spiro[2-benzofuran-1,1′-cyclohexan]-3-one, which was used without further purification. LCMS (ES) m/z 232.2 (M+H)⁺.

To a solution of 4′-(aminomethyl)-3H-spiro[2-benzofuran-1,1′-cyclohexan]-3-one (56 mg, 0.24 mmol) in MeOH (2 mL), was added 2-chloro-4-methyl-3-nitropyridine (46 mg, 0.27 mmol) and Et₃N (0.05 mL, 0.36 mmol). The reaction mixture was heated to reflux for approximately 3 days. Silica gel chromatography (10%-45% EtOAc/hexanes) gave 4′-{[(4-methyl-3-nitropyridin-2-yl)amino]methyl}-3H-spiro[2-benzofuran-1,1′-cyclohexan]-3-one, 32. LCMS (ES) m/z 368.2 (M+H)⁺.

A solution of 32 (31 mg, 0.08 mmol) in EtOH (3 mL) was evacuated and filled with nitrogen three times. Pd/C (100 mg, 10% by weight) was added, and the reaction was again evacuated and filled with nitrogen three times. After a final evacuation, a hydrogen balloon was placed over the reaction. Upon completion, the reaction mixture was filtered through a pad of celite, washing with methanol containing 1% NH₄OH, and concentrated to give 4′-{[(3-amino-4-methylpyridin-2-yl)amino]methyl}-3H-spiro[2-benzofuran-1,1′-cyclohexan]-3-one, which was used without further purification. LCMS (ES) m/z 338.2 (M+H)⁺.

To a solution of 4′-{[(3-amino-4-methylpyridin-2-yl)amino]methyl}-3H-spiro[2-benzofuran-1,1′-cyclohexan]-3-one (28 mg, 0.08 mmol) in DMF, was added 1-hydroxybenzotriazole hydrate (13 mg, 0.08 mmol), 3,3,3-trifluoropropionic acid (0.01 mL, 0.11 mmol), 1-[3-(dimethylamino)propyl]-3-ethylcarbodiimide hydrochloride (24 mg, 0.12 mmol), and Et₃N (0.02 mL, 0.17 mmol). Additional trifluoropropionic acid, EDCI, and Et₃N were added as necessary to force the reaction to completion. Upon completion, the reaction mixture was concentrated in vacuo and loaded directly onto a silica gel column (1%-8% MeOH/CH₂Cl₂). Product was re-purified via silica gel chromatography (30%-80% EtOAc/hexanes) to give 3,3,3-trifluoro-N-(4-methyl-2-{[(3-oxo-3H-spiro[2-benzofuran-1,1′-cyclohexan]-4′-yl)methyl]amino}pyridin-3-yl) propanamide, 33. LCMS (ES) m/z 448.2 (M+H)⁺.

### Experimental for the Preparation of Compound 38 Example 17 in Table C

### N-(2-{[(5′-chloro-2′-oxo-1′,2′-dihydrospiro[cyclohexane-1,3′-indol]4-yl)methyl]amino}-4-methylpyridin-3-yl)-3,3,3-trifluoropropanamide

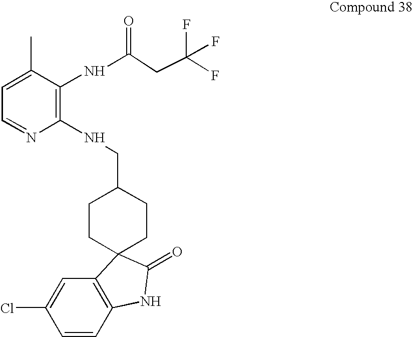

A solution of 19 (1.41 g, 5.13 mmol) in toluene (52 mL) and CH₃CN (5.2 mL) was cooled to 0° C., and 4-chlorophenylhydrazine (879 mg, 6.16 mmol) was added. After approximately forty minutes, the reaction was moved to a 10° C. bath. After thirty minutes, trifluoroacetic acid (1.58 mL, 20.54 mmol) was added, and the reaction was warmed to room temperature. The reaction was moved to a 30° C. bath after 30 minutes, and then the bath was warmed to 50° C. thirty minutes later. After overnight stirring, the reaction mixture was cooled to 0° C., added MeOH (3.47 mL), and followed by NaBH4 (111 mg, 2.93 mmol). After completion, the reaction mixture was quenched with 10 mL of 10% conc. NH4OH, and diluted with EtOAc and washed with saturated NaHCO₃ and brine. The organic phase was dried over sodium sulfate, filtered, and concentrated. Silica gel chromatography (1%-8% MeOH/CH₂Cl₂) gave benzyl [(5′-chlorospiro[cyclohexane-1,3′-indol]4-yl)methyl]carbamate, 34. LCMS (ES) m/z 383.1 (M+H)⁺.

To a solution of 34 (363 mg, 0.95 mmol) in CH₃CN (50 mL) and water (25 mL), was added one equivalent (150 mg, 0.95 mmol) of KMnO₄. The resulting solution was placed halfway into a 50° C. bath, open to the air. Additional equivalents of KMnO₄ (four total) were added over a period of approximately five hours. The reaction mixture was then cooled and diluted with CH₂Cl₂ (500 mL) to get a biphasic mixture. The aqueous layer was extracted with CH₂Cl₂ (200 mL) and the combined organics were washed with water (1×400 mL) and half brine (1×200 mL), dried over sodium sulfate, filtered and concentrated. Silica gel chromatography (20%-60% EtOAc/hexanes) gave benzyl [(5′-chloro-2′-oxo-1′,2′-dihydrospiro[cyclohexane-1,3′-indol]-4-yl)methyl]carbamate, 35. LCMS (ES) m/z 399.1 (M+H)⁺.

HBr gas was bubbled into a solution of 35 (116 mg, 0.29 mmol) in CH₂Cl₂ (6 mL) at 0° C. for approximately five minutes. The reaction mixture sat at 0° C. for several hours before being warmed to room temperature. After sitting at room temperature overnight, several drops (˜10) of HOAc were added to the reaction mixture and a precipitate started to form. After approximately six hours, nitrogen gas was bubbled through the reaction mixture for ten minutes. Diethyl ether (10 mL) was added and nitrogen was again blown through the mixture. The liquid phase was then poured off and replaced with fresh diethyl ether. The same process of washing the precipitate was carried out two additional times. Residual diethyl ether was blown off under a stream of nitrogen and the product placed on the high vacuum pump to give the HBr salt of 4-(aminomethyl)-5′-chlorospiro[cyclohexane-1,3′-indol]-2′(1′H)-one, 36, which was used without further purification. LCMS (ES) m/z 265.1 (M+H−Br)⁺.

To a solution of the HBr salt of 36 (100 mg, 0.29 mmol) and 2-chloro-4-methyl-3-nitropyridine (60 mg, 0.35 mmol) in MeOH (1 mL), was added Et₃N (0.12 mg, 0.87 mmol). The reaction vessel was sealed and the reaction heated to 70° C. After stirring at 70° C. for two days, an additional 1.3 equivalents of 2-chloro-4-methyl-3-nitropyridine and 3 equivalents of Et₃N were added. After an additional two days of stirring at 70° C., the reaction mixture was cooled and concentrated. Silica gel chromatography (20%-70% EtOAc/hexanes) gave 5′-chloro-4-{[(4-methyl-3-nitropyridin-2-yl)amino]methyl}spiro[cyclohexane-1,3′-indol]-2′(1′H)-one, 37. LCMS (ES) m/z 401.1 (M+H)⁺.

To a solution of 37 (80 mg, 0.20 mmol) in EtOAc (2 mL) was added HOAc (0.5 mL). To improve solubility, EtOH (1 mL), EtOAc (1 mL), and HOAc (0.5 mL) were added. Then, zinc dust (196 mg, 3.00 mmol) was added. After approximately forty five minutes, the reaction mixture was partitioned between saturated sodium bicarbonate (40 mL) and EtOAc (100 mL). The organic phase was washed with half brine (1×) and brine (1×), dried over sodium sulfate, filtered, and concentrated. Silica gel chromatography (1%-10% MeOH/CH₂Cl₂ with 10% NH₄OH) gave 4-{[(3-amino-4-methylpyridin-2-yl)amino]methyl}-5′-chlorospiro[cyclohexane-1,3′-indol]-2′(1′H)-one. LCMS (ES) m/z 371.2 (M+H)⁺.

To a solution of 4-{[(3-amino-4-methylpyridin-2-yl)amino]methyl}-5′-chlorospiro[cyclohexane-1,3′-indol]-2′(1′H)-one (58 mg, 0.16 mmol) and 1-hydroxybenzotriazole hydrate (5 mg, 0.03 mmol) in DMF (1.2 mL), was added 3-3-3-trifluroropropionic acid (0.02 mL, 0.19 mmol), Et₃N (0.03 mL, 0.22 mmol), and 1-[3-(dimethylamino)propyl]-3-ethylcarbodiimide hydrochloride (42 mg, 0.22 mmol). After overnight stirring, additional acid (1.2 eq.), Et₃N (1.4 eq.), and EDCI (1.4 eq) were added, and the reaction mixture stirred over the weekend. The reaction mixture was partitioned between water (15 mL) and EtOAc (30 mL). The water layer was extracted with EtOAc (1×). The combined organics were washed with saturated CuSO₄ (1×), half brine (2×), and brine (1×), dried over sodium sulfate, filtered, and concentrated. Silica gel chromatography (1%-10% MeOH/CH₂Cl₂) gave N-(2-{[(5′-chloro-2′-oxo-1′,2′-dihydrospiro[cyclohexane-1,3′-indol]-4-yl)methyl]amino}-4-methylpyridin-3-yl)-3,3,3-trifluoropropanamide, 38. LCMS (ES) m/z 481.2 (M+H)⁺.

### Experimental for the Preparation of Compound Example 18 in Table C

### N-{2-[(3′,4′-dihydrospiro[cyclohexane-1,1′-isochromen]4-ylmethyl)amino]-4-methylpyridin-3-yl}-3,3,3-trifluoropropanamide

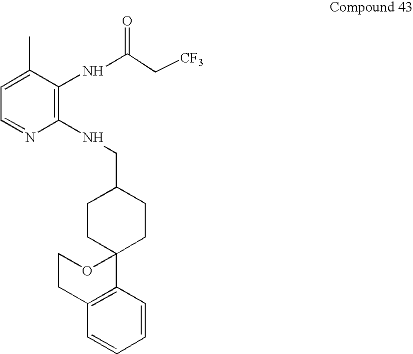

2-(2-bromophenyl)ethanol (5.00 g, 24.87 mmol), tert-butyldimethylsilyl chloride (4.50 g, 29.84 mmol), imidazole (2.54 g, 37.30 mmol), and Et₃N (2.09 mL, 12.43 mmol) were mixed together in DMF (50 mL) and left to stir at room temperature overnight. An additional equivalent of tert-butyldimethylsilyl chloride was added and the reaction was essentially complete after one hour. The reaction mixture was diluted with EtOAc and washed with water (3×100 mL). The aqueous phase was then back extracted with EtOAc (1×100 mL). The combined organics were washed with brine (3×100 mL), dried over sodium sulfate, filtered, and concentrated. Silica gel chromatography (20%-70% CH₂Cl₂/hexanes) gave [2-(2-bromophenyl)ethoxy](tert-butyl)dimethylsilane.

[2-(2-bromophenyl)ethoxy](tert-butyl)dimethylsilane (6.46 g, 20.49 mmol) was dissolved in THF (20 mL) in an oven dried and nitrogen purged flask. The resulting solution was cooled to −78° C. and t-butyl lithium (18.84 mL of a 1.7M solution in pentane) was added dropwise. In a separate oven dried and purged flask, 11 (2.00 g, 12.81 mmol) was dissolved in THF (20 mL) and cooled to −78° C. This solution was then cannulated into the t-butyl lithium solution. After approximately one hour, the reaction was quenched with water (20 mL), warmed to room temperature, and extracted with EtOAc (1×50 mL). The organic phase was washed with water (2×50 mL), and brine (1×50 mL). The combined aqueous layers were back extracted with EtOAc (1×50 mL). The combined organics were dried over sodium sulfate, filtered, and concentrated. Silica gel chromatography, eluting first with 20% CH₂Cl₂/hexanes then with 65% EtOAc/hexanes gave 8-[2-(2-{[tert-butyl(dimethyl)silyl]oxy}ethyl)phenyl]-1,4-dioxaspiro[4.5]decan-8-ol, 39.

To a solution of 39 (100 mg, 0.26 mmol) in CH₃CN (1 mL) at 0° C. was added trifluoroacetic acid (1.0 mL, 12.98 mL). After approximately 1.5 hours, the reaction was quenched with saturated NaHCO₃ (5 mL) and extracted with EtOAc. The organic phase was washed with saturated NaHCO₃ (1×50 mL) and water (1×50 mL). The aqueous layers were back extracted with EtOAc. The combined organics were then dried over sodium sulfate, filtered, and concentrated. Silica gel chromatography (10%-50% EtOAc/hexanes) gave 3″,4″-dihydrodispiro[1,3-dioxolane-2,1′-cyclohexane-4′,1″-isochromene]. LCMS (ES) m/z 217.2 (M+H)⁺.

To a solution of 3″,4″-dihydrodispiro[1,3-dioxolane-2,1′-cyclohexane-4′,1″-isochromene] (500 mg, 1.92 mmol) in 6 mL of dioxane/water (6:1), was added p-toluenesulfonic acid (992 mg, 5.76 mmol), and the resulting solution heated to 50° C. The reaction mixture stirred at 50° C. for approximately two days before it was cooled, diluted with water, and extracted with EtOAc (2×50 mL). The organic phases were combined, dried over sodium sulfate, filtered, and concentrated. Silica gel chromatography (1%-10% ether/CH₂Cl₂) gave 3′,4′-dihydro-4H-spiro[cyclohexane-1,1′-isochromen]-4-one, 40. LCMS (ES) m/z 217.2 (M+H)⁺.

To a solution of 40 (300 mg, 1.39 mmol) in nitromethane (10 mL), was added a catalytic amount of ethylene diamine. The resulting solution was heated to reflux in a Dean-Stark apparatus for approximately three hours. The reaction mixture was cooled and loaded directly onto a silica gel column (1%-7% ether/CH₂Cl₂) to give 4-(nitromethylene)-3′,4′-dihydrospiro[cyclohexane-1,1′-isochromene]. LCMS (ES) i/z 260.2 (M+H)⁺.

A solution of 4-(nitromethylene)-3′,4′-dihydrospiro[cyclohexane-1,1′-isochromene] (267 mg, 1.03 mmol) in MeOH (4 mL) was purged with nitrogen for ten minutes and raney nickel (−4 mL of a slurry in water) was added. The resulting suspension was purged with nitrogen for an additional ten minutes. A hydrogen balloon was placed over the reaction, it was purged with H₂ for ten minutes, and then stirred under a hydrogen atmosphere for two hour. The reaction mixture was filtered through a pad of celite, washing with MeOH. The filtrate was concentrated and purified via silica gel chromatography (0%-1% MeOH/CHCl₃ flushing with 40% MeOH/CH₂Cl₂ with 1% NH₄OH) to give (3′,4′-dihydrospiro[cyclohexane-1,1′-isochromen]-4-ylmethyl)amine, 41. LCMS (ES) m/z 232.2 (M+H)⁺.

2-chloro-4-methyl-3-nitropyridine (36 mg, 0.21 mmol), 41 (40 mg, 0.17 mmol), Et₃N (0.07 mL, 0.52 mmol), and THF (1 mL) were mixed together in a sealed tube and heated to 70° C. After overnight stirring, the reaction mixture was heated to 90° C. for three hours. The temperature was increased to 100° C., and the reaction mixture stirred overnight. The reaction mixture was cooled and concentrated in vacuo. Silica gel chromatography (0%-1% ether/CH₂Cl₂) gave N-(3′,4′-dihydrospiro[cyclohexane-1,1′-isochromen]-4-ylmethyl)-4-methyl-3-nitropyridin-2-amine. LCMS (ES) m/z 368.2 (M+H)⁺.

A solution of N-(3′,4′-dihydrospiro[cyclohexane-1,1′-isochromen]4-ylmethyl)₄-methyl-3-nitropyridin-2-amine (39 mg, 0.11 mmol) in MeOH (5 mL) was purged with nitrogen for ten minutes, and Raney nickel (−2 mL of a slurry in water) was added. The resulting suspension was purged with nitrogen for an additional ten minutes. A hydrogen balloon was placed over the reaction mixture, it was purged with H₂ for ten minutes, and then stirred under a hydrogen atmosphere approximately twenty minutes. The reaction mixture was filtered through a pad of celite, washing with MeOH. Concentration of the filtrate gave N-2-(3′,4′-dihydrospiro[cyclohexane-1,1′-isochromen]4-ylmethyl)-4-methylpyridine-2,3-diamine, 42, which was used without further purification. LCMS (ES) m/z 338.2 (M+H)⁺.

To a solution of 42 (20 mg, 0.06 mmol) in CH₂Cl₂ (1 mL), was added Et₃N (0.04 mL, 0.30 mmol), 1-[3-(dimethylamino)propyl]-3-ethylcarbodiimide hydrochloride (34 mg, 0.18 mmol), 1-hydroxy-7-azabenzotriazole (24 mg, 0.18 mmol), and 3,3,3-trifluoropropionic acid (0.006 mL, 0.07 mmol). After overnight stirring, the reaction mixture was washed with water (1×). The water layer was then back extracted with CH₂Cl₂ (1×), and the combined organic phases were dried over sodium sulfate, filtered, and concentrated. Silica gel chromatography (1%-4% MeOH/CH₂Cl₂) gave N-{2-[(3′,4′-dihydrospiro[cyclohexane-1,1′-isochromen]4-ylmethyl)amino]-4-methylpyridin-3-yl}-3,3,3-trifluoropropanamide, 43. LCMS (ES) m/z 448.2 (M+H)⁺.

### Experimental for the Preparation of Compound 50 Example 34 in Table C

### 3,3,3-trifluoro-N-{4-methyl-2-[(3H-spiro[2-benzofuran-1,1′-cyclohexan]-4′-ylmethyl) amino]pyridin-3-yl}propanamide

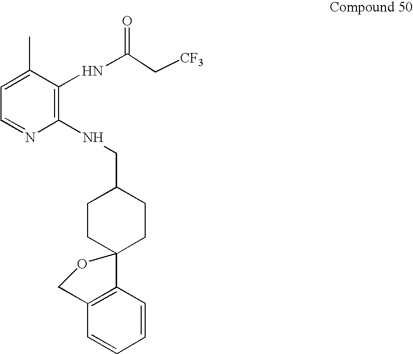

To a solution of 2-bromobenzyl alcohol (10.0 g, 53.46 mmol) in THF (100 mL) at 0° C., NaH (6.42 g of a 60% dispersion in mineral oil) was added slowly. When the bubbling had subsided, chloromethyl methyl ether (6.09 mL, 80.20 mmol) was added. The reaction was allowed to warm to room temperature and stirred overnight. The reaction was quenched with water and partitioned between EtOAc and water. The organic phase was washed with water (1×50 mL) and brine (1×50 mL). The aqueous layers were then back extracted with EtOAc (1×50 mL). The combined organics were dried over sodium sulfate, filtered, and concentrated to give 1-bromo-2-[(methoxymethoxy)methyl]benzene, which was used without further purification.

In an oven dried and purged flask, 1-bromo-2-[(methoxymethoxy)methyl]benzene (9.62 g, 41.62 mmol) was dissolved in THF (50 mL), cooled to −78° C., and t-butyl lithium (37.66 mL of a 1.7M solution in pentane) was added. The resulting solution was cannulated into a solution of 11 (5.00 g, 32.01 mmol) in THF (50 mL) at −78° C. After approximately fifteen minutes, the reaction was warmed to room temperature and quenched with water. The reaction mixture was partitioned between EtOAc and water. The organic phase was washed with water (1×50 mL) and brine (1×50 mL). The aqueous layers were then back extracted with EtOAc (1×50 mL). The combined organics were dried over sodium sulfate, filtered, and concentrated. The crude product was purified via silica gel chromatography (1%-10% ether/CH₂Cl₂ with MeOH added to flush). Re-purification via flash chromatography on silica gel (10%-40% EtOAc/hexanes) gave 8-{2-[(methoxymethoxy)methyl]phenyl}-1,4-dioxaspiro[4.5]decan-8-ol, 44.

To a solution of 44 (9.00 g, 29.18 mmol) in CH₃CN (100 mL) at 0° C., was added trifluroroacetic acid (20 mL, 259.6 mmol). The reaction mixture was allowed to warm to room temperature and stirred overnight. The reaction mixture was neutralized with saturated NaHCO₃ and washed with water (1×50 mL). The aqueous layer was then back extracted with EtOAc (1×50 mL). The combined organic phases were dried over sodium sulfate, filtered, and concentrated to give 3H-dispiro[2-benzofuran-1,1′-cyclohexane-4′,2″-[1,3]dioxolane], which was used without further purification.

To solution of 3H-dispiro[2-benzofuran-1,1′-cyclohexane-4′,2″-[1,3]dioxolane] (6.81 g, 27.65 mmol) in 70 mL of dioxane/water (6:1), was added p-toluenesulfonic acid (14.28 g, 82.94 mmol). The resulting solution was heated to 50° C. and stirred overnight. The reaction mixture was cooled, diluted with EtOAc, and washed with water (1×50 mL) and brine (1×50 mL). The aqueous layers were then back extracted with EtOAc (3×50 mL). The combined organics were dried over sodium sulfate, filtered, and concentrated. Silica gel chromatography (5%-20% EtOAc/hexanes) gave both 4′H,5H-spiro[2,4-benzodioxepine-1,1′-cyclohexan]4′-one, 45, and 3H,4′H-spiro[2-benzofuran-1,1′-cyclohexan]-4′-one, 46.

In an oven dried and purged flask, (methoxymethyl)triphenylphosphonium chloride (1.95 g, 5.69 mmol) was dissolved in THF (10 mL) and potassium t-butoxide (721 mg, 6.43 mmol) was added. In a separate oven dried and purged flask, 46 (1.00 g, 4.94 mmol) was dissolved in THF (20 mL). The resulting solution was cannulated into the phosphonium salt solution. After approximately thirty minutes, the reaction was quenched with water, diluted with EtOAc, and washed with water (1×50 mL) and brine (1×50 mL). The aqueous layers were then back extracted with EtOAc (1×50 mL). The combined organics were dried over sodium sulfate, filtered, and concentrated to give 4′-(methoxymethylene)-3H-spiro[2-benzofuran-1,1′-cyclohexane], which was used without further purification. LCMS (ES) m/z 231.3 (M+H)⁺.

To a solution of 4′-(methoxymethylene)-3H-spiro[2-benzofuran-1,1′-cyclohexane] (1.18 g, 5.12 mmol) in 15 mL of dioxane/water (6:1), was added p-toluenesulfonic acid (1.01 g, 5.89 mmol), and the resulting solution heated to 50° C. The reaction mixture stirred at 50° C. for approximately two hours before it was cooled, diluted with EtOAc, and washed with water (1×50 mL) and brine (1×50 mL). The aqueous phases were back extracted with EtOAc (1×50 mL). The combined organic phases were dried over sodium sulfate, filtered, and concentrated. Silica gel chromatography (5%-20% EtOAc/hexanes) gave 3H-spiro[2-benzofuran-1,1′-cyclohexane]4′-carbaldehyde.

To a solution of 3H-spiro[2-benzofuran-1,1′-cyclohexane]4′-carbaldehyde (309 mg, 1.43 mmol) in THF (3 mL) at 0° C., was added sodium borohydride (5.72 mL of a 0.5M solution in 2-methoxy ethyl ether). The reaction mixture was warmed to room temperature. Upon completion, the reaction was quenched with saturated NaHCO₃, diluted with EtOAc, and washed with water (1×50 mL) and brine (1×50 mL). The aqueous phases were back extracted with EtOAc (1×50 mL). The combined organics were dried over sodium sulfate, filtered, and concentrated to give 3H-spiro[2-benzofuran-1,1′-cyclohexan]-4′-ylmethanol, 47, which was used without further purification. LCMS (ES) m/z 219.4 (M+H)⁺.

To a solution of 47 (310 mg, 1.42 mmol) in CH₂Cl₂ (6 mL) at 0° C., Et₃N (0.59 mL, 4.26 mmol) and then methane sulfonyl chloride (0.22 mL, 2.84 mmol) were added. The reaction mixture was warmed to room temperature and stirred overnight. The next morning, the reaction mixture was washed with water (1×50 mL) and brine (1×50 mL). The aqueous layers were back extracted with EtOAc (1×50 mL). The combined organics were dried over sodium sulfate, filtered, and concentrated to give 3H-spiro[2-benzofuran-1,1′-cyclohexan]-4′-ylmethyl methanesulfonate, which was used without further purification.

3H-spiro[2-benzofuran-1,1′-cyclohexan]4′-ylmethyl methanesulfonate (420 mg, 1.42 mmol) and sodium azide (281 mg, 4.25 mmol) were mixed together in DMF (3 mL) and heated to 80° C. overnight. The reaction mixture was cooled, diluted with EtOAc, and washed with water (3×50 mL) and brine (1×50 mL). The organic phase was dried over sodium sulfate, filtered, and concentrated. Silica gel chromatography (5%-20% EtOAc/hexanes) gave 3H-spiro[2-benzofuran-1,1′-cyclohexan]4′-ylmethyl azide. LCMS (ES) m/z 216.3 (M+H−N₂)⁺.

A solution of 3H-spiro[2-benzofuran-1,1′-cyclohexan]-4′-ylmethyl azide (100 mg, 0.41 mmol) in 10 mL of EtOAc/EtOH (1:1) was purged with nitrogen for ten minutes, and Pd/C (76 mg, 10% by weight) was added. The resulting suspension was purged with nitrogen for an additional fifteen minutes. A hydrogen balloon was placed over the reaction, it was purged with hydrogen, and stirred overnight under an atmosphere of hydrogen. The next morning, the reaction mixture was filtered through a pad of celite, washing with MeOH containing trace amounts of Et₃N. Concentration of the filtrate gave (3H-spiro[2-benzofuran-1,1′-cyclohexan]4′-ylmethyl)amine, 48, which was used without further purification. LCMS (ES) m/z 218.4 (M+H)⁺.

In a sealed tube, 2-chloro-4-methyl-3-nitropyridine (77 mg, 0.45 mmol), 48 (88 mg, 0.41 mmol), and Et₃N (0.17 mL, 1.22 mmol) were mixed together in THF (10 mL) and heated to 100° C. The reaction mixture stirred at 100° C. for approximately four days before it was cooled and concentrated. Silica gel chromatography (10%-30% EtOAc/hexanes) gave 4-methyl-3-nitro-N-(3H-spiro[2-benzofuran-1,1′-cyclohexan]-4′-ylmethyl)pyridin-2-amine, 49. LCMS (ES) m/z 354.3 (M+H)⁺.

A solution of 49 (100 mg, 0.28 mmol) in EtOH (10 mL) was purged with nitrogen for ten minutes. Raney nickel (˜2 mL of a slurry in water) was added and the resulting slurry was purged with nitrogen for fifteen minutes. A hydrogen balloon was placed over the reaction mixture, the reaction mixture was purged with hydrogen for ten minutes, and the reaction complete after an additional twenty minutes of stirring. The reaction mixture was filtered through a pad of celite, washing with MeOH that contained trace amounts of Et₃N. Concentration of the filtrate gave 4-methyl-N-2-(3H-spiro[2-benzofuran-1,1′-cyclohexan]4′-ylmethyl)pyridine-2,3-diamine, which was used without further purification. LCMS (ES) m/z 324.4 (M+H)⁺.

To a solution of 4-methyl-N-2-(3H-spiro[2-benzofuran-1,1′-cyclohexan]-4′-ylmethyl)pyridine-2,3-diamine (65 mg, 0.20 mmol) in CH₂Cl₂ (3 mL), was added Et₃N (0.14 mL, 1.01 mmol), 1-[3-(dimethylamino)propyl]-3-ethylcarbodiimide hydrochloride (116 mg, 0.60 mmol), 1-hydroxy-7-azabenzotriazole (82 mg, 0.60 mmol), and 3,3,3-trifluoropropionic acid (0.02 mL, 0.24 mmol. After overnight stirring, the reaction mixture was washed with water (1×). The water layer was then back extracted with CH₂Cl₂ (1×). The combined organic phases were dried over sodium sulfate, filtered, and concentrated. Silica gel chromatography (20%-60% EtOAc/hexanes) gave 3,3,3-trifluoro-N-{4-methyl-2-[(3H-spiro[2-benzofuran-1,1′-cyclohexan]4′-ylmethyl)amino]pyridin-3-yl}propanamide, 50. LCMS (ES) m/z 434.3 (M+H)⁺.

### Experimental for the Preparation of Compound 55 Example 38 in Table C

### 2-cyano-N-{4-methyl-2-[(spiro[cyclohexane-1,1′-isochromen]-4-ylmethyl)amino]pyridin-3-yl}acetamide

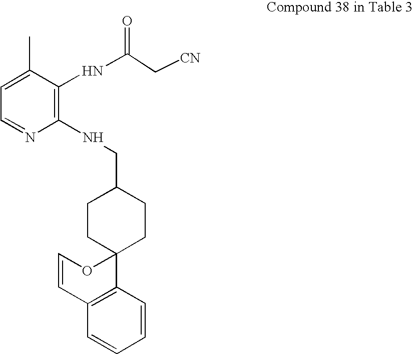

To a solution of 39 (6.62 g, 16.86 mmol) in THF (50 mL) was added tetrabutylammonium fluoride (33.7 mL of a 1M solution in THF). After overnight stirring, the reaction mixture was diluted with EtOAc and washed with water (1×50 mL) and brine (1×50 mL). The aqueous layers were back extracted with EtOAc (1×50 mL) and the combined organic phases were dried over sodium sulfate, filtered, and concentrated. Silica gel chromatography (10%-40% EtOAc/hexanes) gave 8-[2-(2-hydroxyethyl)phenyl]-1,4-dioxaspiro[4.5]decan-8-ol.

To a solution of oxalyl chloride (0.71 mL, 8.08 mmol) in CH₂Cl₂ (15 mL) at −78° C., was added DMSO (0.65 mL, 9.16 mmol) dropwise over a period of fifteen minutes. After approximately thirty minutes, a solution of 8-[2-(2-hydroxyethyl)phenyl]-1,4-dioxaspiro[4.5]decan-8-ol (1.5 g, 5.39 mmol) dissolved in CH₂Cl₂ (15 mL) at −78° C. was cannulated into the oxalyl chloride solution. The resulting solution stirred for approximately fifteen minutes and Et₃N was added (3.00 mL, 21.56 mmol). The reaction mixture was then warmed to 0° C. Upon completion, the reaction mixture was diluted with CH₂Cl₂ and washed with saturated NH₄Cl (1×50 mL), water (1×50 mL), and brine (1×50 mL). The organic phase was dried over sodium sulfate, filtered, and concentrated to give dispiro[1,3-dioxolane-2,1′-cyclohexane-4′,1″-isochromene], 51, which was used without further purification. LCMS (ES) m/z 259.3 (M+H)⁺.

To a solution of 51 (1.16 g, 4.49 mmol) in 7 mL of dioxane/water (6:1), was added p-toluenesulfonic acid (928 mg, 5.39 mmol). The resulting solution was heated to 50° C. for approximately forty five minutes, cooled, and purified directly by flash chromatography on silica gel (10%-40% EtOAc/hexanes) to give 4H-spiro[cyclohexane-1,1′-isochromen]4-one. LCMS (ES) m/z 215.3 (M+H)⁺.

In an oven dried and purged flask, (methoxymethyl)triphenylphosphonium chloride (1.35 g, 3.93 mmol) was dissolved in THF (5 mL) and potassium t-butoxide (4.44 mL of a 1M solution in THF) was added. In a separate oven dried and purged flask, 4H-spiro[cyclohexane-1,1′-isochromen]-4-one (732 mg, 3.42 mmol) was dissolved in THF (5 mL). The resulting solution was added to the phosphonium salt solution via syringe. After approximately one hour, the reaction was quenched with water, diluted with EtOAc, and washed with water (1×50 mL) and brine (1×50 mL). The aqueous layers were then back extracted with EtOAc (1×50 mL). The combined organics were dried over sodium sulfate, filtered, and concentrated to give 4-(methoxymethylene)spiro[cyclohexane-1,1′-isochromene], which was used without further purification. LCMS (ES) m/z 243.3 (M+H)⁺.

To a solution of 4-(methoxymethylene)spiro[cyclohexane-1,1′-isochromene] (823 mg, 3.40 mmol) in 6 mL of dioxane/water (6:1), was added p-toluenesulfonic acid (877 mg, 5.09 mmol). The resulting solution was heated to 50° C. for approximately one hour. The reaction mixture was then cooled, diluted with EtOAc and washed with water (1×50 mL) and brine (1×50 mL). The aqueous layers were back extracted with EtOAc (1×50 mL) and the combined organics dried over sodium sulfate, filtered, and concentrated. Silica gel chromatography (10%-40% EtOAc/hexanes) gave spiro[cyclohexane-1,1′-isochromene]4-carbaldehyde, 52. LCMS (ES) m/z 229.3 (M+H)⁺.

A solution of 52 (510 mg, 2.23 mmol) in THF (10 mL) was cooled to 0° C. and sodium borohydride (6.70 mL of a 0.5M solution in 2-methoxyethyl ether) was added. The resulting solution was warmed to room temperature. Upon completion, the reaction was quenched with saturate NaHCO₃ and washed with water (1×50 mL) and brine (1×50 mL). The aqueous layers were back extracted with EtOAc (1×50 mL), and the combined organics were dried over sodium sulfate, filtered, and concentrated to give spiro[cyclohexane-1,1′-isochromen]-4-ylmethanol, which was used without further purification. LCMS (ES) m/z 231.4 (M+H)⁺.

To a solution of spiro[cyclohexane-1,1′-isochromen]-4-ylmethanol (514 mg, 2.23 mmol) in CH₂Cl₂ (5 mL), Et₃N (0.93 mL, 6.70 mmol) was added. The resulting solution was cooled to 0° C. and methanesulfonyl chloride (0.35 mL, 4.46 mmol) was added. The reaction mixture was then allowed to warm to room temperature and stirred overnight. The reaction mixture was washed with water (1×50 mL) and brine (1×50 mL) and the aqueous layers then back extracted with CH₂Cl₂ (1×50 mL). The combined organic phases were dried over sodium sulfate, filtered, and concentrated to give spiro[cyclohexane-1,1′-isochromen]4-ylmethyl methanesulfonate, which was used without further purification. LCMS (ES) m/z 309.3 (M+H)⁺.

Spiro[cyclohexane-1,1′-isochromen]-4-ylmethyl methanesulfonate (680 mg, 2.21 mmol) and sodium azide (437 mg, 6.62 mmol) were mixed together in DMF (3 mL) and heated to 80° C. After approximately four hours, the reaction mixture was cooled, diluted with EtOAc, and washed with water (1×50 mL) and brine (1×50 mL). The aqueous layers were back extracted with EtOAc (1×50 mL) and the combined organics dried over sodium sulfate, filtered, and concentrated. Silica gel chromatography (5%-20% EtOAc/hexanes) gave 4-(azidomethyl)spiro[cyclohexane-1,1′-isochromene], 53. LCMS (ES) m/z 228.3 (M+H−N₂)⁺.

To a solution of 53 (387 mg, 1.52 mmol) in THF (10 mL), was added triphenyl phosphine (477 mg, 1.82 mmol). After stirring at room temperature for three hours, 25 mL of water was added to the reaction mixture. After overnight stirring, the reaction mixture was diluted with EtOAc and washed with saturated NaHCO₃ (1×50 mL), water (1×50 mL), and brine (1×50 mL). The aqueous layers were back extracted with EtOAc and the combined organics dried over sodium sulfate, filtered, and concentrated. Silica gel chromatography (1%-5% MeOH/CH₂Cl₂ with 1% Et₃N) gave (spiro[cyclohexane-1,1′-isochromen]-4-ylmethyl)amine. LCMS (ES) m/z 230.4 (M+H)⁺.

2-chloro-4-methyl-3-nitropyridine (248 mg, 1.44 mmol), (spiro[cyclohexane-1,1′-isochromen]4-ylmethyl)amine (300 mg, 1.31 mmol), Et₃N (0.55 mL, 3.92 mmol), and THF (10 mL) were mixed together in a sealed tube and heated to 100° C. overnight. The next morning, approximately 5 mL of the solvent were removed, the reaction vessel re-sealed, and heated again to 100° C. for four hours. An additional equivalent of 2-chloro-4-methyl-3-nitropyridine was added and the reaction mixture stirred overnight. The next morning the reaction mixture was cooled and concentrated in vacuo. Silica gel chromatography (5%-20% EtOAc/hexanes) gave 4-methyl-3-nitro-N-(spiro [cyclohexane-1,1′-isochromen]4-ylmethyl)pyridin-2-amine, 54. LCMS (ES) m/z 366.3 (M+H)⁺.

A solution of 54 (166 mg, 0.45 mmol) in EtOH (10 mL) was purged with nitrogen for ten minutes, and raney nickel (˜2 mL of a slurry in water) was added. The resulting suspension was purged with nitrogen for an additional fifteen minutes. A hydrogen balloon was placed over the reaction, the reaction mixture was purged with hydrogen, and the reaction stirred under a hydrogen atmosphere for approximately two hours. Filtration through a pad of celite, washing with MeOH containing trace amounts of Et₃N, and concentration of the filtrate gave 4-methyl-N-2-(spiro[cyclohexane-1,1′-isochromen]4-ylmethyl)pyridine-2,3-diamine, which was used without further purification. LCMS (ES) m/z 336.3 (M+H)⁺.

To a solution of 4-methyl-N-2-(spiro[cyclohexane-1,1′-isochromen]-4-ylmethyl)pyridine-2,3-diamine (75 mg, 0.22 mmol) in CH₂Cl₂ (3 mL), was added Et₃N (0.16 mL, 1.12 mmol), 1-[3-(dimethylamino)propyl]-3-ethylcarbodiimide hydrochloride (129 mg, 0.67 mmol), 1-hydroxy-7-azabenzotriazole (91 mg, 0.67 mmol), and 3,3,3-trifluoropropionic acid (0.016 mL, 0.27 mmol). After overnight stirring, the reaction mixture was washed with water (1×). The water layer was then back extracted with CH₂Cl₂ (1×), and the combined organic phases were dried over sodium sulfate, filtered, and concentrated. Silica gel chromatography (1%-7% MeOH/CH₂Cl₂) gave 2-cyano-N-{4-methyl-2-[(spiro[cyclohexane-1,1′-isochromen]-4-ylmethyl)amino]pyridin-3-yl}acetamide, 55. LCMS (ES) m/z 403.3 (M+H)⁺.

TABLE A through TABLE E below, provide compounds of Formula I, II and III that have been prepared by the method described above. The binding affinity for BK receptor B1, in the table ranges from 0.4 nM to 10 nM.

TABLE A

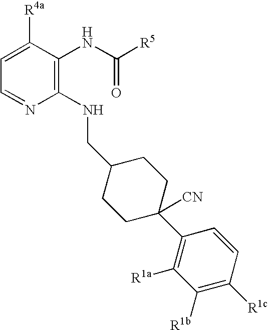

| Example | R^{4a} | R⁵ | R^{1a} | R^{1b} | R^{1c} | Stereo | MS(ES) |
|---|---|---|---|---|---|---|---|
| 1 | H | CH₂CF₃ | Cl | H | H | | 451 |
| 2 | CH₃ | CH₂CF₃ | H | H | H | cis/trans | 4312 |
| 3 | H | CH₂CN | Cl | H | H | cis/trans | 408 |
| 4 | CH₃ | CH₂CF₃ | Cl | H | H | cis/trans | 465.3 |
| 5 | CH₃ | CH₂CN | Cl | H | H | cis/trans | 422.2 |
| 6 | H | CH₂CF₃ | H | Cl | Cl | trans | 485.3 |
| 7 | CH₃ | CH₂CF₃ | H | H | H | trans | 431.4 |
| 8 | H | CH₂CN | H | Cl | Cl | | 442.3 |
| 9 | CH₃ | CH₂CN | H | H | H | trans | 388.4 |
| 10 | H | CH₂CF₃ | H | H | H | cis | 417.3 |
| 11 | H | CH₂CN | H | H | H | cis | 374.3 |
| 12 | CH₃ | CH₂CN | H | H | H | cis | 388.3 |
| 13 | CH₃ | CH₂CF₃ | H | H | H | cis | 431.4 |
| 14 | H | CH₂CF₃ | Cl | Cl | H | | 485.2 |
| 15 | H | CH₂CN | Cl | Cl | H | | 442.3 |
| 16 | H | CH₂CF₃ | F | F | H | | 435.4 |
| 17 | H | CH₂CN | H | F | H | | 392.4 |
| 18 | H | CH₂CF₃ | F | H | H | | 435.4 |
| 19 | H | CH₂CN | F | H | H | | 392.4 |
| 20 | H | CH₂CF₃ | CF₃ | H | H | | 485.4 |
| 21 | H | CH₂CN | CF₃ | H | H | | 442.4 |
| 22 | H | CH₂CF₃ | F | F | H | | 453.4 |
| 23 | H | CH₂CN | F | F | H | | 410.4 |
| 24 | H | CH₂CF₃ | H | H | F | cis/trans | 435.4 |
| 25 | H | CH₂CN | H | H | F | cis/trans | 392.4 |
| 26 | H | CH₂CF₃ | OCH₃ | F | H | | 483.4 |
| 27 | H | 5-isoxazole | H | H | F | | 420.4 |
| 28 | H | CH₂CF₃ | CF₃ | H | H | | 485.4 |
| 29 | H | CH₂CN | H | CF₃ | H | | 442.4 |
| 30 | H | CH₂CF₃ | OCF₃ | H | H | | 501.4 |
| 31 | H | CH₂CN | OCF₃ | H | H | | 458.4 |
| 32 | H | CH₂CF₃ | H | CO₂CH₃ | H | | 475.5 |
| 33 | H | CH₂CN | H | CO₂CH₃ | H | | 432.3 |
| 34 | Cl | CH₂CF₃ | H | F | H | cis/trans | 469.7 |
| 35 | H | CH₂CN | H | morpholine | H | cis/trans | 459.8 |

TABLE B

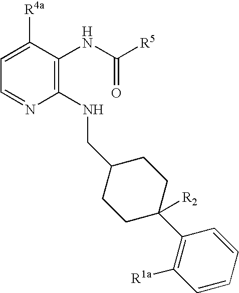

| Example 1 | R^{4a} | R⁵ | R₂ | R^{1a} | Stereo | MS(ES) |
|---|---|---|---|---|---|---|
| 1 | H | CH₂CF₃ | CO₂CH₃ | H | cis/trans | 450.1 |
| 2 | H | CH₂CF₃ | CH₂OH | H | cisltrans | 422.2 |
| 3 | CH₃ | CH₂CF₃ | CH₃ | H | cis/trans | 420.2 |
| 4 | CH₃ | CH₂CF₃ | phenyl | H | | 482.2 |
| 5 | CH₃ | CH₂CN | phenyl | H | | 439.2 |
| 6 | H | CH₂CF₃ | O(CO)CH₃ | H | cis/trans | 450.3 |
| 7 | H | CH₂CN | O(CO)CH₃ | H | cis/trans | 407.4 |
| 8 | H | CH₂CF₃ | OH | H | cis | 408.4 |
| 9 | CH₃ | CH₂CN | OH | H | cis | 365.3 |
| 10 | H | CH₂CF₃ | OH | H | trans | 408.4 |
| 11 | H | CH₂CN | OH | H | trans | 365.4 |
| 12 | H | CH₂CF₃ | 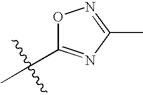 | H | cis/trans | 474.5 |
| 13 | H | CH₂CF₃ | 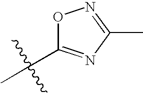 | H | cis/trans | 431.4 |
| 14 | H | CH₂CF₃ | C(O)NH₂ | H | | 435.4 |
| 15 | CH₃ | CH₂CN | H | CO₂CH₃ | | 421.2 |

TABLE C

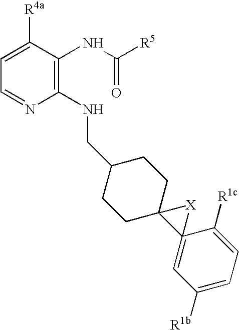

| Example | R^{4a} | R⁵ | R^{1b} | R^{1c} | X | Stereo | MS(ES) |
|---|---|---|---|---|---|---|---|
| 1 | CH₃ | CH₂CN | H | H | CH₂NS(O)₂CH₃ | | 468.2 |
| 2 | CH₃ | CH₂CF₃ | H | H | CH₂NS(O)₂CH₃ | | 511.2 |
| 3 | CH₃ | CH₂CN | H | H | CH₂NS(O)₂CH₃ | | 468.2 |
| 4 | CH₃ | CH₂CF₃ | H | H | CH₂NS(O)₂CH₃ | | 511.2 |
| 5 | CH₃ | CH₂CN | H | H | CH₂NC(O)CF₃ | | 486.2 |
| 6 | CH₃ | CH₂CF₃ | H | H | CH₂NC(O)CF₃ | | 529.3 |
| 7 | CH₃ | CH₂CN | H | H | CH₂NC(O)OCH₃ | | 448.3 |
| 8 | CH₃ | CH₂CF₃ | H | H | CH₂NC(O)OCH₃ | | 491.3 |
| 9 | CH₃ | CH₂CF₃ | H | H | CH₂NC(O)OC(CH₃)₃ | | 533.3 |
| 10 | CH₃ | CH₂CF₃ | H | H | CH₂NH₂ | | 433.2 |
| 11 | CH₃ | CH₂CF₃ | H | H | CH₂NC(O)CH₃ | | 475.3 |
| 12 | CH₃ | CH₂CF₃ | H | H | CH₂NCH₃ | | 447.2 |
| 13 | CH₃ | CH₂CF₃ | Cl | H | CH₂NH₂ | | 467.2 |
| 14 | H | CH₂CF₃ | Cl | H | CH₂NH₂ | | 453.2 |
| 15 | CH₃ | CH₂CF₃ | H | H | OC(O) | cis/trans | 448.2 |
| 16 | CH₃ | CH₂CF₃ | H | H | CH₂NCH₂CF₃ | | 515.3 |
| 17 | CH₃ | CH₂CF₃ | Cl | H | C(O)NH | | 481.2 |
| 18 | CH₃ | CH₂CF₃ | H | H | OCH₂CH₂ | | 448.2 |
| 19 | CH₃ | CH₂CF₃ | Cl | H | OC(O) | trans | 482.1 |
| 20 | CH₃ | CH₂CN | Cl | H | OC(O) | trans | 439.1 |
| 21 | CH₃ | CH₂CF₃ | Cl | H | OC(O) | Cis | 482.1 |
| 22 | CH₃ | CH₂CN | Cl | H | OC(O) | cis | 439.2 |
| 23 | CH₃ | CH₂CF₃ | H | H | CH₂CH₂ | cis/trans | 432.2 |
| 24 | CH₃ | CH₂CF₃ | H | H | OC(O) | cis | 448.2 |
| 25 | CH₃ | CH₂CN | H | H | OCH₂CH₂ | | 405.2 |
| 26 | CH₃ | CH₂CF₃ | H | Cl | OC(O) | trans | 482.1 |
| 27 | CH₃ | CH₂CF₃ | H | Cl | OC(O) | cis | 482.1 |
| 28 | CH₃ | 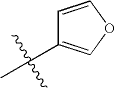 | H | Cl | OC(O) | cis | 466.1 |
| 29 | CH₃ | CH₂CN | H | Cl | OC(O) | cis | 439.1 |
| 30 | H | CH₂CN | H | Cl | OC(O) | cis | 425.1 |
| 31 | CH₃ | n-propyl | H | Cl | OC(O) | | 442.2 |
| 32 | H | CH₂CF₃ | H | Cl | OC(O) | cis | 468.2 |
| 33 | CH₃ | 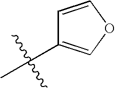 | H | H | OCH₂CH₂ | | |
| 34 | CH₃ | CH₂CF₃ | H | H | OCH₂ | cis/trans | 434.3 |
| 35 | CH₃ | CH₂CN | H | H | OCH₂ | cis/trans | 391.3 |
| 36 | CH₃ | CH₂CF₃ | H | H | OCH₂OCH₂ | | 464.4 |
| 37 | CH₃ | CH₂CN | H | H | OCH₂OCH₂ | | 421.3 |
| 38 | CH₃ | CH₂CN | H | H | OCHCH | cis/trans | 403.3 |
| 39 | CH₃ | CH₂CF₃ | H | H | OCHCH | cis/trans | 446.4 |
| 40 | H | CH₂CF₃ | H | F | OC(O) | cis | 452.3 |
| 41 | H | CH₂CF₃ | H | F | OC(O) | trans | 452.4 |
| 42 | H | CH₂CN | H | F | OC(O) | cis | 409.3 |
| 43 | H | CH₂CN | H | F | OC(O) | trans | 409.2 |
| 44 | Cl | CH₂CF₃ | H | F | OC(O) | cis | 486.3 |
| 45 | H | CH₂CF₃ | H | Cl | NHC(O) | cis | 467.4 |
| 46 | H | CH₂CF₃ | H | Cl | NHC(O) | trans | 467.6 |

TABLE D

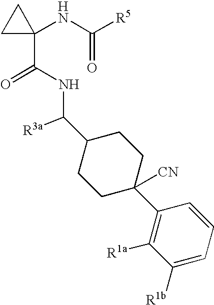

| | | | | | | |
|---|---|---|---|---|---|---|
| Example | R⁵ | R^{3a} | R^{1a} | R^{1b} | Stereo | MS(ES) |

| | | | | | |
|---|---|---|---|---|---|
| 1 | CH₂CF₃ | H | Cl | Cl | 476.3 |
| 2 | CH₂CN | H | Cl | Cl | 433.3 |
| 3 | CH₂CF₃ | H | CF₃ | H | 476.7 |
| 4 | CH₂CN | H | CF₃ | H | 433.4 |
| 5 | CH₂CF₃ | H | H | CO₂CH₃ | 466.7 |
| 6 | CH₂CN | H | H | CO₂CH₃ | 423.7 |
| 7 | CH₂CF₃ | H | CF₃ | F | 494.6 |
| 8 | 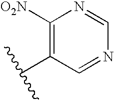 | H | CF₃ | H | 488.6 |
| 9 | 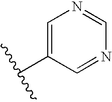 | H | CF₃ | H | 472.6 |
| 10 | 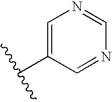 | H | F | H | 422.6 |
| 11 | 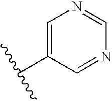 | CH₃ | F | H | 436.6 |
| 12 | CH₂CF₃ | CH₃ | F | H | 440.6 |
| 13 | CH₂CF₃ | H | F | H | 426.6 |
| 14 | C(O)CH₃ | H | CF₃ | H | 408.3 |
| 15 | C(O)NHCH₃ | H | CF₃ | H | 423.2 |
| 16 | C(O)NHC(O)OCH₃ | H | CF₃ | H | 467.1 |

TABLE E

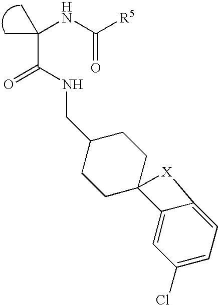

| Ex- am- ple | R⁵ | 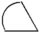 | X | Stereo | MS(ES) |
|---|---|---|---|---|---|
| 1 | CH₂CF₃ | cyclopropyl | OC(O) | cis | 459.6 |
| 2 | CH₂CF₃ | cyclopropyl | OC(O) | trans | 459.6 |
| 3 | 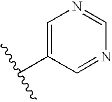 | cyclobutyl | CH₂NH | cis | 454.3 |

1. A compound of Formula I, Formula II or Formula III: 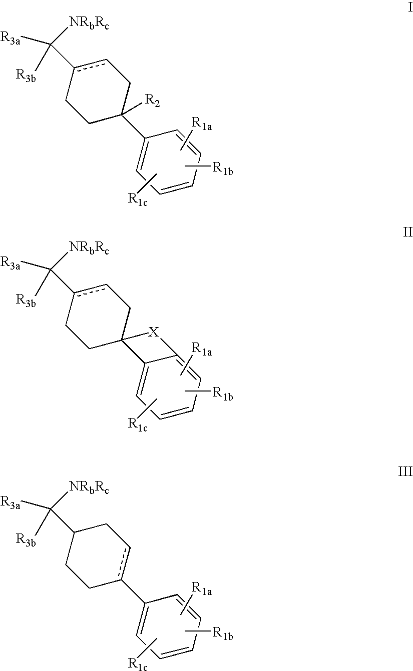
or a pharmaceutically acceptable salt thereof wherein
R^{1a}, R^{1b} and R^{1c} are each selected from
(1) hydrogen,
(2) —C₁₋₈ alkyl, optionally substituted with 1, 2, 3, 4 or 5 groups independently selected from halogen, nitro, cyano, —COR^{a}, —CO₂R^{a}, —CONR^{d}R^{e}, —OR^{a}, —OC(O)R^{a}, —SOₘR^{a′}, —NR^{d}R^{e}, —NR^{d}C(O)R^{a}, —NR^{d}SO₂R^{a′}, —NR^{d}CO₂R^{a},
(3) —C₃₋₈ cycloalkyl,
(4) —C₂₋₈ alkenyl optionally substituted with CO₂R^{a},
(5) halogen,
(6) cyano,
(7) nitro,
(8) —NR^{d}R^{e},
(9) —NR^{d}C(O)R^{a},
(10) —NR^{d}CO₂R^{a},
(11) —NR^{d}C(O)NR^{d}R^{e},
(12) —NR^{d}C(O)NR^{d}CO₂R^{a},
(13) —NR^{d}SO₂R^{a′},
(14) —CO₂R^{a},
(15) —COR^{a},
(16) —C(O)NR^{d}R^{e},
(17) —C(O)NHOR^{a},
(18) —C(═NOR^{a})R^{a},
(19) —C(═NOR^{a})NR^{d}R^{e},
(20) —OR^{a},
(21) —OC(O)R^{a},
(22) —S(O)ₘR^{a}′, wherein R^{a}′ is a non-hydrogen group selected from R^{a},
(23) —SO₂NR^{d}R^{e},
(24) substituted or unsubstituted heterocycle (such as oxadiazole, tetrazole, triazole, pyrazole, oxazole, isoxazole, thiazole, 4,5-dihydro-oxazole, 4,5-dihydro-1,2,4-oxadiazol-5-one), wherein the heterocycle is (a) a 5-membered aromatic ring having a ring heteroatom selected from N, O and S, and optionally having up to 3 additional ring nitrogen atoms wherein said ring is optionally benzo-fused; (b) a 6-membered aromatic ring containing from 1 to 3 ring nitrogen atoms and N-oxides thereof, wherein said ring is optionally benzo-fused; and (c) a 5- or 6-membered non-aromatic heterocyclic ring and wherein the substituents are 1, 2 or 3 groups independently selected from C₁₋₄alkyl optionally substituted with 1, 2 or 3 halogen atoms, —OR^{a}, or —OC(O)R^{a},
with the proviso that not more than one of R^{1a}, R^{1b} and R^{1c} is a heterocycle;
R² is selected from
(1) H,
(2) —C₁₋₆ alkyl optionally substituted with 1, 2 or 3 halogen atoms,
(3) —C₃₋₇ cycloalkyl optionally containing 1 or 2 ring members selected from O and N−R^{d} group,
(4) —(CH₂)ₙ—C(O)—C₁₋₆ alkyl optionally substituted with 1, 2 or 3 halogen atoms,
(5) —(CH₂)ₙOR^{a},
(6) —(CH₂)ₙS(O)ₘR^{a′},
(7) —(CH₂)ₙNR^{d}R^{e},
(8) —(CH₂)ₙC(O)OR^{a},
(9) —(CH₂)ₙOCOR^{a′},
(10) —(CH₂)ₙNR^{d}C(O)R^{a′},
(11) —(CH₂)ₙNS(O)ₘR^{a′},
(12) —(CH₂)ₙC(O)NR^{d}R^{e},
(13) —(CH₂)ₙ CN,
(14) —(CH₂)ₙ-AR, wherein AR is selected from benzene, naphthalene, indole, indoline, is optionally substituted with 1, 2, 3 or 4 groups independently selected from halogen, C₁₋₄ alkyl optionally substituted with 1, 2, 3, 4 or 5 halogen atoms,
(15) —(CH₂)ₙ—NO2,
(16) —(CH₂)ₙ-heterocycle, wherein the heterocycle is an optionally substituted (a) 5-membered ring having a ring heteroatom selected from N, O and S, and optionally having up to 3 additional ring nitrogen atoms wherein said ring is optionally benzo-fused; (b) 6-membered ring containing from 1 to 3 ring nitrogen atoms and N-oxides thereof, wherein said ring is optionally benzo-fused; and (c) 5- or 6-membered non-aromatic heterocyclic ring, and wherein the substituents on the heterocycle are with 1, 2 or 3 groups independently selected from —C₁₋₄ alkyl optionally substituted with 1 to 5 halogen atoms, —OR^{a} or —OC(O)R^{a};
R^{3a} and R^{3b} are selected from
(1) H and
(2) —C₁₋₄ alkyl optionally substituted with 1, 2 or 3 halogen atoms;
R^{a} is independently selected from:
(1) H,
(2) —C₁₋₆ alkyl optionally substituted with 1, 2 or 3 halogen atoms,
(3) —C₃₋₇ cycloalkyl ring, the ring optionally containing 1 or 2 ring members selected from O and N—R^{d} group,
(4) —C(O)—C₁₋₆ alkyl optionally substituted with 1, 2 or 3 halogen atoms,
(5) AR1, wherein AR1 is selected from benzene, pyridine, thiophene, naphthalene, indole, indoline, pyrimidine, imidazole, optionally substituted with 1, 2, 3 or 4 groups independently selected from halogen, C₁₋₄ alkyl optionally substituted with 1, 2, 3, 4 or 5 halogen atoms, hydroxy, C₁₋₄ alkoxy optionally substituted with 1, 2, 3, 4 or 5 halogen atoms,
(6) nitro,
(7) cyano, and
(8) NR^{d}R^{e};
R^{a′} is independently selected from:
(1) —C₁₋₆ alkyl optionally substituted with 1, 2 or 3 halogen atoms,
(2) —C₃₋₇ cycloalkyl optionally containing ring 0 and/or N—R^{d} group,
(3) —C(O)—C₁₋₆ alkyl optionally substituted with 1, 2 or 3 halogen atoms,
(4) AR1, wherein AR1 is selected from benzene, pyridine, thiophene, naphthalene, indole, indoline, pyrimidine, imidazole optionally substituted with 1, 2, 3 or 4 groups independently selected from halogen, C₁₋₄ alkyl optionally substituted with 1, 2, 3, 4, or 5 halogen atoms,
(5) hydroxy,
(6) —C₁₋₄ alkoxy optionally substituted with 1, 2, 3, 4 or 5 halogen atoms,
(7) nitro,
(8) cyano, and
(9) —NR^{d}R^{e};
R^{b} and R^{f} are each independently selected from:
(1) H,
(2) —C₁₋₆ alkyl optionally substituted with 1, 2 or 3 halogen atoms,
R^{c} is independently selected from:
(1) hydrogen,
(2) —C₁₋₆ alkyl optionally substituted with 1, 2 or 3 halogen atoms,
(3) —C₃₋₇ cycloalkyl optionally containing 1 or 2 ring members selected from O and N—R^{e} group,
(4) 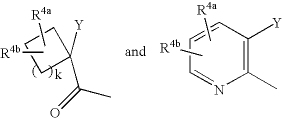
R^{4a} and R^{4b} are independently selected form
(1) H,
(2) halogen, and
(3) —C₁₋₄ alkyl optionally substituted with 1, 2, 3 or 4 groups selected from halogen, —OR^{a}, —OC(O)R^{a}, —S(O)ₘR^{a′}, —OS(O)₂R^{a′}, and —NR^{d}R^{e};
X is selected from
(1) —(CH₂)ₙN[S(O)₂R^{a′}](CH₂)ₙ—,
(2) —(CH₂)ₙ N[C(O)R^{a}](CH₂)ₙ—,
(3) —(CH₂)ₙN[C(O)OR^{a}](CH₂)ₙ—,
(4) —(CH₂)ₙNR^{d}(CH₂)ₙ—,
(5) —(CH₂)ₙOC(O)(CH₂)ₙ—,
(6) —(CH₂)ₙC(O)NR^{d}(CH₂)ₙ—,
(7) —(CH₂)ₙC(O)O(CH₂)ₙ—,
(8) —(CH₂)ₙNR^{d}C(O)(CH₂)ₙ—,
(9) —(CH₂)ₙNS(O)₂(CH₂)ₙ—,
(10) —(CH₂)ₙS(O)₂N(CH₂)ₙ—,
(11) —(CH₂)ₙS(O)₂O(CH₂)ₙ—,
(12) —(CH₂)ₙOS(O)₂(CH₂)ₙ—,
(13) —(CH₂)ₙOS(O)₂O(CH₂)ₙ—,
(14) —(CH₂)ₙNS(O)₂O(CH₂)ₙ—,
(15) —(CH₂)ₙOS(O)₂N(CH₂)ₙ—,
(16) —(CH₂)ⱼ—,
(17) —(CH₂)ₙO(CH₂)ₙ—,
(18) —(CH₂)ₙO(CH₂)ⱼO(CH₂)ₙ—, and
(19) —(CH₂)ₙCH═CH(CH₂)ₙ—;
Y is selected from
(1) —NR^{b}C(O)R⁵,
(2) —NR^{d}C(O)NR^{d}R^{e},
(3) —OC(O)NR^{d}R^{e},
(4) —NR^{d}S(O)₂NR^{d}R^{e},
(5) —NR^{d}C(O)OR^{a},
R^{d} and R^{e} are each independently selected from:
(1) H,
(2) —C₁₋₆ alkyl optionally substituted with 1 to 3 halogen atoms,
(3) —C₃₋₇ cycloalkyl optionally containing ring 0 and/or N—R^{b} group
(4) —(CH₂)ₙNR^{b}R^{f},
(5) —(CH₂)ₙOR^{a},
(6) —(CH₂)ₙS(O)ₘR^{a′},
(7) —(CH₂)ₙC(O)OR^{a},
(8) —(CH₂)ₙC(O)NR^{b}R^{f}
(9) —C(O)R^{f},
(10) —S(O)₂R^{f}
(11) AR2,
(12) -AR2-C₁₋₄alkyl,
(13) —C₁₋₄-alkyl-AR2,
wherein AR2, -AR2-C₁₋₄alkyl, and —C₁₋₄-alkyl-AR2 are optionally substituted with 1, 2, 3 or 4 groups independently selected from halogen, —C₁₋₄ alkyl optionally substituted with 1, 2, 3, 4 or 5 halogen atoms, hydroxy, —C₁₋₄ alkoxy optionally substituted with 1, 2, 3, 4 or 5 halogen atoms, nitro, cyano and —NR^{b}R^{c}, and wherein AR2 is selected from benzene, pyridine, thiophene, naphthalene, indene, indan, thiodiazole, benzofuran, indole, indoline, benzothiophene, pyrimidine, triazine, thioazole, isoxazole, oxazole, benzimidazole, imidazole;
R⁵ is selected from
(1) —C₁₋₆ alkyl optionally substituted with 1, 2, 3, 4 or 5 groups independently selected from halogen, nitro, cyano, —OR^{a}, —SR^{a}, —COR^{a}, —SO₂R^{a′}, —CO₂R^{a}, —OC(O)R^{a}, —NR^{d}R^{e}, —NR^{d}C(O)R^{a}, —NR^{d}C(O)₂R^{a}, —C(O)NR^{d}R^{e}, —C₃₋₈ cycloalkyl,
(2) —C₃₋₈ cycloalkyl optionally substituted with 1 to 5 groups independently selected from halogen, nitro, cyano and phenyl,
(3) —C₃₋₆ alkynyl,
(4) —C₂₋₆ alkenyl optionally substituted with hydroxyethyl,
(5) —(CH₂)ₙ-AR4 optionally substituted with 1 to 3 groups independently selected from halogen, nitro, cyano, —OR^{a}, —SR^{a}, —C(O)₂R^{a}, —C₁₋₄ alkyl and —C₁₋₃ haloalkyl, wherein AR4 is selected from phenyl, 3,4-methylenedioxyphenyl and naphthyl;
(6) —(CH₂)ₙ-heterocycle optionally substituted with 1, 2 or 3 groups independently selected from halogen, nitro, cyano, OR^{a}, SR^{a}, C₁₋₄ alkyl and C₁₋₃ haloalkyl wherein said heterocycle is selected from (a) a 5-membered ring having a ring heteroatom selected from N, O and S, and optionally having up to 3 additional ring nitrogen atoms wherein said ring is optionally benzo-fused; (b) a 6-membered ring containing from 1 to 3 ring nitrogen atoms and N-oxides thereof, wherein said ring is optionally benzo-fused; and (c) a 5- or 6-membered non-aromatic heterocyclic ring selected from tetrahydrofuranyl, 5-oxotetrahydrofuranyl, 2-oxo-2H-pyranyl, 6-oxo-1,6-dihydropyridazinyl,
(7) —C(O)₂R^{a}, and
(8) —C(O)NR^{d}R^{e};
n is 0, 1, 2, 3 or 4;
m is 0, 1 or 2;
j is 1, 2, 3 or 4,
k is 0 or 1.

2. A compound according to claim 1 wherein
R^{c} is independently selected from:
(1) hydrogen,
(2) —C₁₋₆ alkyl optionally substituted with 1, 2 or 3 halogen atoms,
(3)

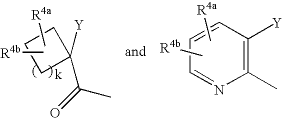

3. A compound according to claim 1 wherein
X is selected from:
(1) —CH₂NH,
(2) —OC(O)—,
(3) —C(O)NH—,
(4) —OCH₂CH₂—,
(5) —OC(O)—,
(6) —OCH₂—,
(7) —OCH₂OCH₂—,
(8) —OCHCH—, and
(9) —NHC(O)—.

4. A compound according to claim 1 wherein
Y is —NR^{b}C(O)R⁵.

5. A compound according to claim 1 for the Formula I 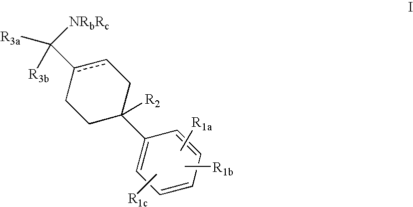
and pharmaceutically acceptable salts thereof wherein R^{b} is H and R^{c} is:

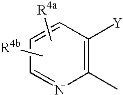

6. A compound according to claim 5 of Formula Ia 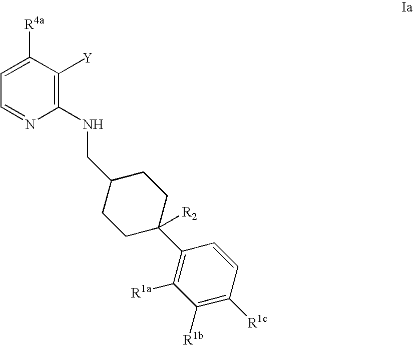
and pharmaceutically acceptable salts.

7. A compound according to claim 6 wherein:
R² is H, CN, OH, CH₃, CH₂OH, C(O)NH₂, CO₂CH₃, OC(O)CH₃, phenyl and oxadiazole,
R^{a4} is selected from hydrogen and CH₃,
R^{1a}, R^{1b} and R^{1c} are each independently selected from the group consisting of hydrogen, halo, CF₃, OCH₃, OCF₃, CO₂CH₃ and morpholine,
Y is NHC(O)R⁵, and
R⁵ is selected from the group consisting of —CH₂CF₃, —CH₂CN and isoxazole.
Within this subgenus there is a class of compounds wherein:
R² is H or CN.

8. A compound according to claim 1 of Formula I 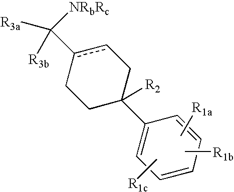
and pharmaceutically acceptable salts thereof wherein Rc is:

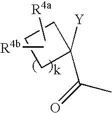

9. A compound according to Claim 8 of Formula Ib

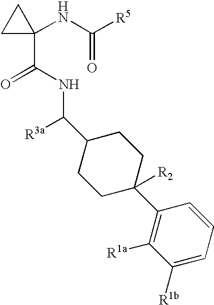

10. A compound according to claim 9 wherein:
R² is CN,
R^{1a} and R^{1b} are each independently selected from the group consisting of hydrogen, halo, —CF₃, —OCH₃, —OCF₃ and —CO₂CH₃,
R^{3a} is selected from the group consisting of hydrogen and methyl, and
R⁵ is selected from the group consisting of —CH₂CF₃, —CH₂CN, —C(O)CH₃, —C(O)NHCH₃, —C(O)NHC(O)OCH₃, pyridyl optionally substituted with NO₂, isoxazole, and pyrimidine.

11. A compound according to claim 1 of Formula Ib

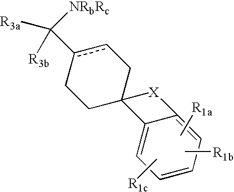

12. A compound according to claim 11 of the Formula Ib′ and Ib″

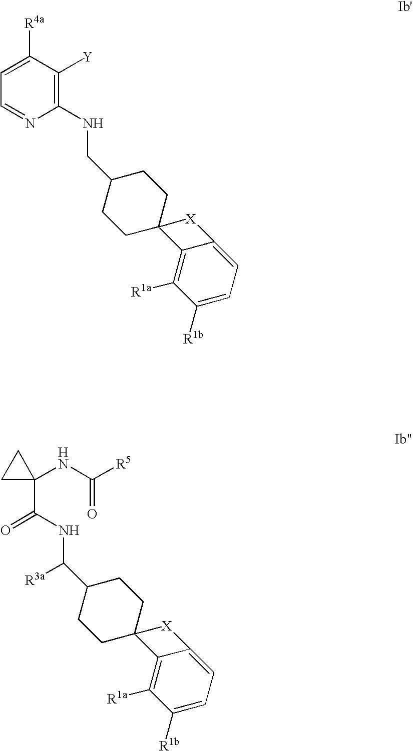

13. A compound according to claim 12 wherein
R^{1a} and R^{1b} are each independently selected from the group consisting of hydrogen and halo;
R³ is hydrogen;
R^{4a} is hydrogen or —CH₃;
Y is NHC(O)R⁵;
R⁵ is selected from the group consisting of —CH₂CF₃, —CH₂CN, furan and pyrimidine; and
X is selected from:
(1) —CH₂NH,
(2) —OC(O)—,
(3) —C(O)NH—,
(4) —OCH₂CH₂—,
(5) —OC(O)—,
(6) —OCH₂—,
(7) —OCH₂OCH₂—,
(8) —OCHCH—, and
(9) —NHC(O)—.

14. A compound selected from the group consisting of methyl 1-phenyl-4-[({3-[(3,3,3-trifluoropropanoyl)amino]pyridin-2-yl}amino)methyl]cyclohexanecarboxylate, 3,3,3-trifluoro-N-(2-{[(4-hydroxy-4-phenylcyclohexyl)methyl]amino}pyridin-3-yl)propanamide, 3,3,3-trifluoro-N-[4-methyl-2-({[1′-(methylsulfonyl)-1′,2′-dihydrospiro[cyclohexane-1,3′-indol]-4-yl]methyl}amino)pyridin-3-yl]propanamide, 3,3,3-trifluoro-N-[4-methyl-2-({[1′-(trifluoroacetyl)-1′,2′-dihydrospiro[cyclohexane-1,3′-indol]-4-yl]methyl}amino)pyridin-3-yl]propanamide, N-{2-[(1′,2′-dihydrospiro[cyclohexane-1,3′-indol]-4-ylmethyl)amino]-4-methylpyridin-3-yl}-3,3,3-trifluoropropanamide, 3,3,3-trifluoro-N-(4-methyl-2-{[(3-oxo-3H-spiro[2-benzofuran-1,1′-cyclohexan]4′-yl)methyl]amino}pyridin-3-yl)propanamide, N-(2-{[(5′-chloro-2′-oxo-1′,2′-dihydrospiro[cyclohexane-1,3′-indol]-4-yl)methyl]amino}-4-methylpyridin-3-yl)-3,3,3-trifluoropropanamide, N-{2-[(3′,4′-dihydrospiro[cyclohexane-1,1′-isochromen]-4-ylmethyl)amino]4-methylpyridin-3-yl}-3,3,3-trifluoropropanamide, 3,3,3-trifluoro-N-{4-methyl-2-[(3H-spiro[2-benzofuran-1,1′-cyclohexan]-4′-ylmethyl)amino]pyridin-3-yl}propanamide, and 2-cyano-N-{4-methyl-2-[(spiro[cyclohexane-1,1′-isochromen]-4-ylmethyl)amino]pyridin-3-yl}acetamide, or a paharmaceutically acceptable salt thereof.

15. A pharmaceutical composition comprising a compound according to claim 1 or a pharmaceutically acceptable salt thereof; and a pharmaceutically acceptable carrier.

16. A method of treatment or prevention of pain and inflammation comprising a step of administering, to a subject in need of such treatment or prevention, an effective amount of a compound according to claim 1 or a pharmaceutically acceptable salt thereof.
